(19) United States
(12) Patent Application Publication
(10) Pub. No.: US 2004/0110768 A1
(43) Pub. Date: Jun. 10, 2004

(54) Bombesin receptor antagonists

(76) Inventors: Michael Higginbottom, Cambridgeshire (GB); Martyn Clive Pritchard, Cambridgeshire (GB); Herman Thijs Stock, Cambridgeshire (GB)

Correspondence Address: PFIZER INC., PATENT DEPARTMENT, MS8260-1611, EASTERN POINT ROAD, GROTON, CT, 06340, US

(21) Appl. No.: 10/416,251

(22) PCT Filed: Nov. 16, 2001
(86) PCT No.: PCT/EP01/14402

(30) Foreign Application Priority Data
Nov. 17, 2000 (GB) 0028146.9

(51) Int. Cl. A61K 31/506; A61K 31/44; A61K 31/4172; A61K 31/381; A61K 31/34

(52) U.S. Cl. 514/256; 514/357; 514/365; 514/400; 514/408; 514/438; 514/471; 544/242; 546/336; 548/204; 548/338.1; 548/567; 549/76; 549/494

(57) ABSTRACT

Bombesin receptor antagonists are provided which are compounds of formula (I) or pharmaceutically acceptable salts thereof:

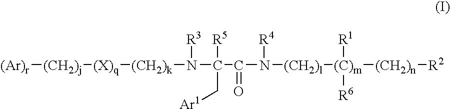

wherein j, k, l, m, n, q, r, Ar, Ar¹, R¹, R², R³, R⁴, R⁵, R⁶ and X are as defined in the description.

The compounds of the invention have an affinity for the BB₁ receptor and some of them also have affinity for the BB₂ receptor. Accordingly they may be useful for the diagnosis, prevention, or treatment of male and female sexual dysfunction. They can also be used in the diagnosis, prevention or treatment of anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders or pruritus.

### FIELD OF THE INVENTION

The present invention relates to chemical compounds that are bombesin receptor antagonists, to methods for the manufacture of the above compounds and to pharmaceutical compositions containing the above compounds. It also relates to the use of the above compounds in the manufacture of medicaments for the prophylaxis or treatment of a variety of disorders in animals (including humans). It further relates to methods for administration of the above compounds to patients for the prophylaxis or treatment of a variety of disorders.

### BACKGROUND TO THE INVENTION

Bombesin is a 14-amino acid peptide originally isolated from the skin of the European frog Bombina bombina (Anastasi A., et al., Experientia, 1971;27:166). It belongs to a class of peptides which share structural homology in their C-terminal decapeptide region (Dutta A. S., Small Peptides; Chemistry, Biology, and Clinical Studies, Chapter 2, pp 66-82). At present, two mammalian bombesin-like peptides have been identified (Battey J., et al., TINS, 1991;14:524), the decapeptide neuromedin B (NMB) and a 23-residue amino acid, gastrin-releasing peptide (GRP). Bombesin-like immunoreactivity has been detected in mammalian brain (Braun M., et al., Life. Sci., 1978;23:2721) and the GI tract (Walsh J. H., et al., Fed. Proc. Fed. Am. Soc. Exp. Biol., 1979;38:2315). This, together with studies measuring mRNA levels in rat brain (Battey J., et al., TINS, 1991;14:524), points to the widespread distribution of both NMB and GRP in mammalian peripheral and central nervous systems. NMB and GRP are believed to mediate a variety of biological actions via acting upon the corresponding bombesin receptors (for review, see WO 98/07718).

Bombesin evokes a number of central effects, e.g. feeding, scratching, and peripheral effects e.g. contraction of rat oesophagus, secretion of gastrin, through actions at a heterogeneous population of receptors (for review, see Battey J. and Wada E., Trends Neurosci., 1991 ;14:524-528). The BB, receptor binds neuromedin B (NMB) with higher affinity than gastrin-related peptide (GRP) and neuromedin C (NMC) and BB₂ receptors bind GRP and NMC with greater affinity than NMB. More recently evidence has emerged of two more receptor subtypes denoted BB₃ and BB₄ but due to limited pharmacology, little is known of their function at present. BB₁ and BB₂ receptors have a heterogeneous distribution within the central nervous system indicating that the endogenous ligands for these receptors may differentially modulate neurotransmission. Among other areas, BB₁ receptors are present in the ventromedial hypothalamus (Ladenheim EE et al, Brain Res., 1990; 537:233-240).

Both males and females can suffer from sexual dysfunction. Sexual dysfunctions are relatively common in the general population (see O'Donohue W, et al, Clin. Psychol. Rev. 1997; 17: 537-566). The disorder may relate to seeking sexual behaviour (proceptivity) and/or to acceptance of sexual behaviour, accompanied by sexual arousal (receptivity). The prevalence of sexual problems is higher in populations receiving medicaments, in particular antidepressants and antihypertensives. A need for pharmacotherapy for sexual dysfunction is increasing, but there has been very little research effort directed at finding drugs to treat sexual dysfunction.

A component of male sexual dysfunction results from mechanical disorder(s), resulting in an inability to achieve penile erection or ejaculation. Treatment has been revolutionised by the unexpected discovery that cGMP PDE inhibitors, e.g. pyrazolo[4,3-d]pyrimidin-7-ones were useful in the treatment of erectile dysfunction and could be administered orally. One such compound that is currently being manufactured is sildenafil (Viagra). A second component of male sexual dysfunction is psychogenic disorders. Psychogenic disorders are also more prevalent in female sexual dysfunction. Thirty to 50% of American women complain of sexual dysfunction. Ageing, menopause, and decline in circulating oestrogen levels significantly increase the incidence of sexual complaints. Berman J. R. et al. (Int. J. Impot. Res., 1999, 11: S31-38) describe a methodology for evaluating physiologic and subjective components of the female sexual response in the clinical setting and determine the effects of age and oestrogen status on them. In a recent publication (Bonney R. C et al., Scrip's Complete Guide to Women's Healthcare, PJB Publications Ltd, London, April 2000) the causes and management of female sexual dysfunction are discussed, including the use of tibolone (Livial), which is a synthetic steroid that mimics the effects of oestrogen and has been reported to have mild androgenic properties, and the use of testosterone.

WO 98/07718 discloses a class of non-peptide compounds capable of antagonising the effects of NMB and/or GRP at bombesin receptors. The compounds are stated to be useful in treating or preventing a variety of disorders including depression, psychoses, seasonal affective disorders, cancer, feeding disorders, gastrointestinal disorders including colitis, Crohn's disease and inflammatory bowel disease, sleeping disorders, and memory impairment.

WO 00/37462 describes non-peptide NK₁ receptor antagonists useful for treating inflammatory and allergic disorders.

### SUMMARY OF THE INVENTION

We have surprisingly found a further class of bombesin receptor antagonists which are compounds of formula (I) or pharmaceutically acceptable salts thereof:

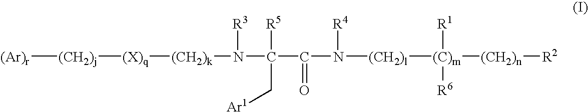

wherein:

j is 0, 1 or 2;

k is 0 or 1;

l is 0, 1, 2, or 3;

m is 0 or 1;

n is 0, 1 or 2;

q is 0 or 1;

r is 0 or 1; when r is 0, Ar is replaced by hydrogen;

Ar is phenyl, pyridyl, pyrimidyl, thienyl, furyl, imidazolyl, pyrrolyl or thiazolyl each unsubstituted or substituted by from 1 to 3 substituents selected from acetyl, alkoxy, alkyl, amino, cyano, halo, hydroxy, nitro, sulfonamido, sulfonyl, —CF₃, —OCF₃, —CO₂H, —CH₂CN, —SO₂CF₃, —CH₂CO₂H and —(CH₂)ₛNR⁷R⁸ wherein s is 0, 1, 2 or 3 and R⁷ and R⁸ are each independently selected from H, straight or branched alkyl of up to 6 carbon atoms, or R⁷ and R⁸, together with the nitrogen atom to which they are linked, can form a 5- to 7-membered aliphatic ring which may contain 1 or 2 oxygen atoms;

R¹ is hydrogen, straight or branched alkyl of up to 6 carbon atoms or cycloalkyl of between 5 and 7 carbon atoms which may contain 1 or 2 nitrogen or oxygen atoms;

R⁶ is hydrogen, methyl or forms with R¹ an aliphatic ring of from 3 to 7 atoms which can contain an oxygen or nitrogen atom, or together with R¹ is a carbonyl group;

Ar¹ is independently selected from Ar or is indolyl or pyridyl-N-oxide;

R³, R⁴, and R⁵ are each independently selected from hydrogen and lower alkyl;

R² is independently selected from Ar or is hydrogen, hydroxy, alkoxy, —NMe₂, —CONR⁹R¹⁰ wherein R⁹ and R¹⁰ are each independently selected from hydrogen, straight or branched alkyl of up to 6 carbon atoms, or R⁹ and R¹⁰ together with the nitrogen atom to which they are linked can form a 5- to 7-membered aliphatic ring which may contain 1 or 2 oxygen or nitrogen atoms, or R² is

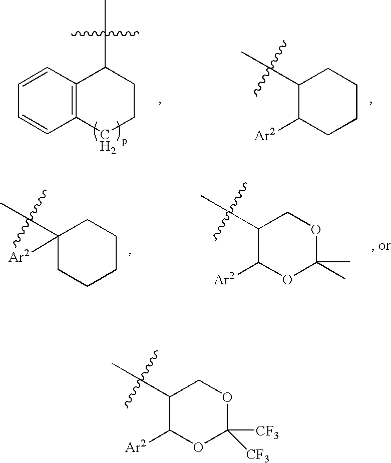

wherein p is 0, 1 or 2 and Ar² is phenyl or pyridyl;

X is a divalent radical derived from any of the following:

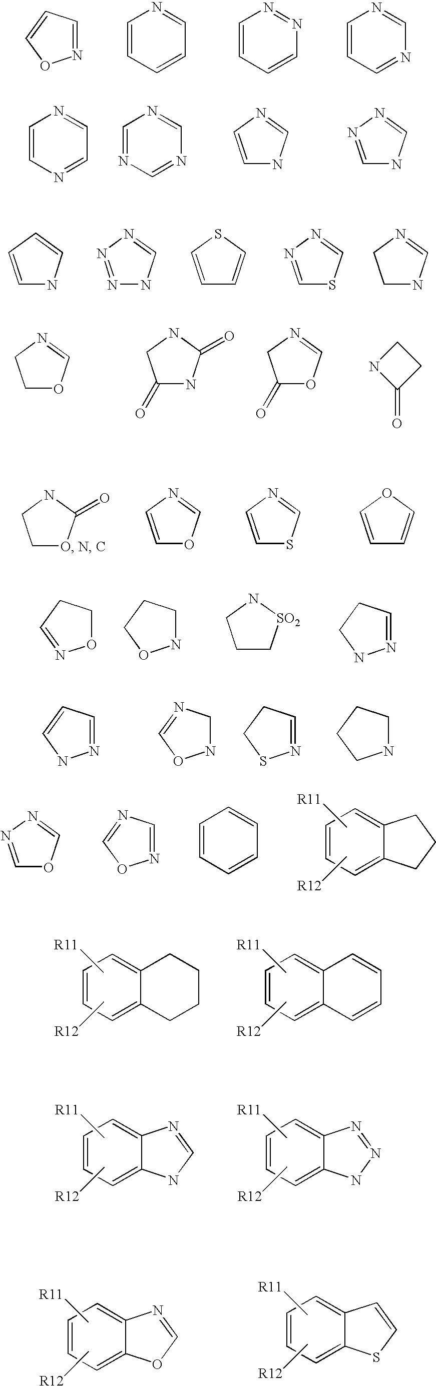

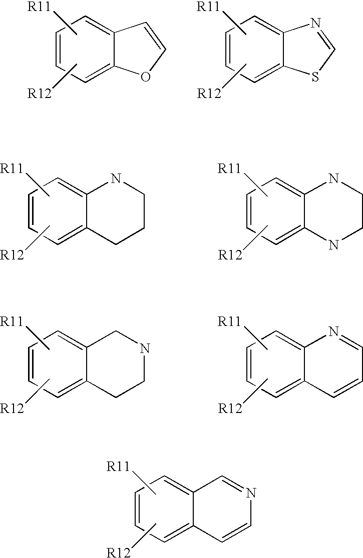

where the ring nitrogen atoms may have lower alkyl groups attached thereto, R¹¹, R¹² are independently selected from H, halogen, hydroxy, alkoxy, acetyl, nitro, cyano, amino, CF₃ and (CH₂)ₜNR¹³R¹⁴ wherein t can be 0 or 1, R¹³ and R¹⁴ are each independently selected from hydrogen, straight or branched alkyl of up to 6 carbon atoms or cycloalkyl of 5 to 7 carbon atoms, containing up to 2 oxygen or nitrogen atoms;

provided that, when Ar¹ is indolyl, then

(i) r is 1 or q is R¹ or

(ii) R⁶ forms with R¹ an aliphatic ring of from 3 to 7 atoms which can contain an oxygen or nitrogen atom, or R⁶ together with R¹ is a carbonyl group.

The compounds of the invention have been evaluated in receptor binding assays which measure their affinity in a cloned human NMB-preferring receptor (BB₁) assay and in a cloned human GRP-preferring receptor (BB₂) assay. It has been found that they have affinity for the BB₁ receptor and some of them also have affinity for the BB₂ receptor. Accordingly they may be useful for the diagnosis, prevention, or treatment of male sexual dysfunction in humans and animals, female sexual dysfunction in humans and animals, anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus.

The invention further provides a method of antagonizing the effects of neuromedin B and/or gastrin-releasing peptide at bombesin receptors which comprises administering a compound of formula (I) to a patient.

The invention further provides a pharmaceutical composition comprising a therapeutically effective amount of a compound of Formula (I) together with at least one pharmaceutically acceptable carrier or excipient.

The invention further provides a method for preventing or treating various diseases amenable to therapy by a bombesin receptor antagonist, including male or female sexual dysfunction, anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus, said method comprising administering to a patient in need of such treatment an effective amount of a bombesin receptor antagonist of Formula (I).

The invention yet further provides the use of a compound of Formula (I) in the manufacture of a medicament for preventing or treating various diseases amenable to therapy by a bombesin receptor antagonist, including male or female sexual dysfunction, anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus.

### BRIEF DESCRIPTION OF FIGURES

FIG. 1: Effect of (S)-3-(1H-Indol-3-yl)-N-[1-(5-methoxy-pyridin-2-yl)-cyclohexyl-methyl]-2-methyl-2-[4-(4-nitro-phenyl)-oxazol-2-ylamino]-propionamide in PEG 200 on female rat sexual proceptivity

FIG. 2: Effect of (S)-3-(1H-Indol-3-yl)-N-[1-(5-methoxy-pyridin-2-yl)-cyclohexyl-methyl]-2-methyl-2-[4-(4-nitro-phenyl)-oxazol-2-ylamino]-propionamide in methyl cellulose on female rat sexual proceptivity.

FIG. 3: Effect of (S)-3-(1H-Indol-3-yl)-N-[1-(5-methoxy-pyridin-2-yl)-cyclohexyl-methyl]-2-methyl-2-[4-(4-nitro-phenyl)-oxazol-2-ylamino]-propionamide in PEG 200 on female rat sexual receptivity.

### DESCRIPTION OF PREFERRED EMBODIMENTS

Definitions

The compounds of Formula (I) are optically active. The scope of the invention therefore also includes:

All stereoisomers of the compounds of Formula (I).

Their solvates, hydrates and polymorphs (different crystalline lattice descriptors) of the compounds of Formula (I).

Pharmaceutical compositions of compounds of Formula (I).

Prodrugs of the compounds of Formula (I) such as would occur to a person skilled in the art, see Bundgaard, et al., Acta Pharm. Suec., 1987;24:233-246.

The lower alkyl groups contemplated by the invention include straight or branched carbon chains of from 1 to 6 carbon atoms, except where specifically stated otherwise. They also include cycloalkyl groups, which are cyclic carbon chains having 3 to 7 carbon atoms, except where specifically stated otherwise, and which may be substituted with from 1 to 3 groups selected from halogens, nitro, straight or branched alkyl, and alkoxy.

The alkoxy groups contemplated by the invention comprise both straight and branched carbon chains of from 1 to 6 carbon atoms unless otherwise stated. Representative groups are methoxy, ethoxy, propoxy, i-propoxy, t-butoxy, and hexoxy.

The term “halogen” is intended to include fluorine, chlorine, bromine, iodine and astatine.

The term “amine” is intended to include free amino, alkylated amines, and acylated amines.

Optical Isomers and Salts

The compounds of Formula (I) all have at least one chiral centre and some have multiple chiral centers depending on their structure. In particular, the compounds of the present invention may exist as diastereomers, mixtures of diastereomers, or as the mixed or the individual optical enantiomers. The present invention contemplates all such forms of the compounds. The mixtures of diastereomers are typically obtained as a result of the reactions described more fully below. Individual diastereomers may be separated from mixtures of the diastereomers by conventional techniques such as column chromatography or repetitive recrystallization. Individual enantiomers may be separated by conventional methods well known in the art such as conversion to a salt with an optically active compound, followed by separation by chromatography or recrystallization and reconversion to the non-salt form.

Where it is appropriate to form a salt, the pharmaceutically acceptable salts include acetate, benzenesulfonate, benzoate, bicarbonate, bitartrate, bromide, calcium acetate, camsylate, carbonate, chloride, citrate, dihydrochloride, edetate, edisylate, estolate, esylate, fumarate, gluceptate, gluconate, glutamate, glycoloylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, mucate, napsylate, nitrate, pamoate (embonate), pantothenate, phosphate/diphosphate, polygalacturonate, salicylate, stearate, subacetate, succinate, sulfate, tannate, tartrate, theoclate, triethiodide, benzathine, chloroprocaine, choline, diethanolamine, ethylenediamine, meglumine, procaine, aluminum, calcium, lithium, magnesium, potassium, sodium, and zinc.

Preferred salts are made from strong acids. Such salts include hydrochloride, mesylate, and sulfate.

Preferred Compounds

A preferred group of compounds is represented by the Formula (II) and includes pharmaceutically acceptable salt thereof:

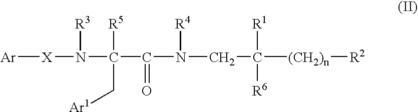

wherein:

n is 0 or 1;

Ar is phenyl or pyridyl which may be unsubstituted or substituted with from 1 to 3 substituents selected from halogen, alkoxy, nitro and cyano;

Ar¹ is independently selected from Ar or is pyridyl-N-oxide or indolyl;

R⁶ forms with R¹ an aliphatic ring of from 3 to 7 atoms which can contain an oxygen or nitrogen atom, or together with R¹ is a carbonyl group;

R² is independently selected from Ar or is hydrogen, hydroxy, alkoxy, dimethylamino, tetrazolyl or —CONR⁹R¹⁰ wherein R⁹ and R¹⁰ are each independently selected from hydrogen or methyl, or R² is any of

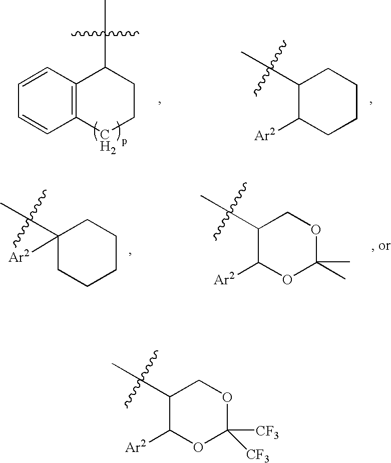

wherein p is 0, 1 or 2, and Ar² is phenyl or pyridyl;

R³, R⁴ and R⁵ are each independently selected from hydrogen and methyl; and

X is selected from:

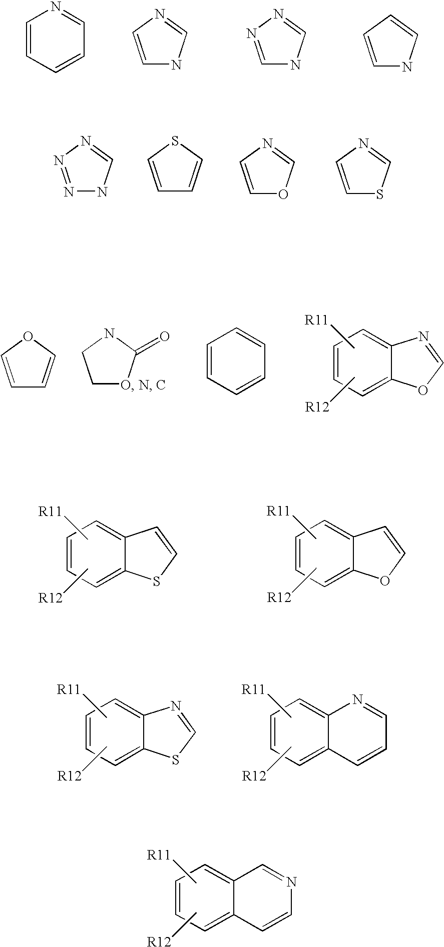

R¹¹ and R¹² being independently selected from H, halogen, hydroxy, alkoxy, acetyl, nitro, cyano, amino, CF₃ and (CH₂)ₜNR¹³R¹⁴ wherein t is 0 or 1 and R¹³ and R¹⁴ are independently selected from hydrogen and methyl.

A further group of preferred compounds has the formula (IIa) or (IIb):

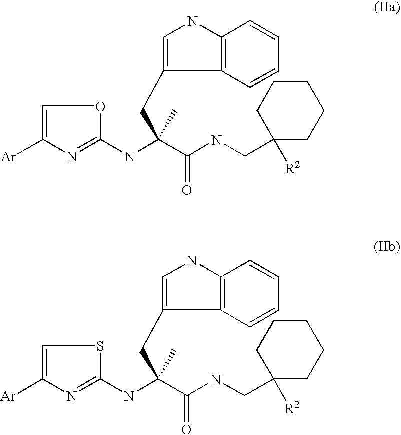

wherein Ar and R² independently represent phenyl or pyridyl which may be unsubstituted or substituted with from 1 to 3 substituents selected from halogen, alkoxy, nitro and cyano,

and pharmaceutically acceptable salts thereof.

Particularly preferred is (S)-3-(1H-Indol-3-yl)-N-[1-(5-methoxy-pyridin-2-yl)-cyclohexylmethyl]-2-methyl-2-[4-(4-nitro-phenyl)-oxazol-2-ylamino]-propionamide (also referred as compound (1)) and its pharmaceutically acceptable salts.

Other preferred compounds are set out below and also included are their pharmaceutically acceptable salts:

(S)-3-(1H-indol-3-yl)-N-(1-methoxymethyl-cyclohexylmethyl)-2-methyl-2-[4-(4-nitro-phenyl)-oxazol-2-ylamino]-propionamide;

(S)-3-(1H-indol-3-yl)-2-methyl-2-[4-(4-nitro-phenyl)-oxazol-2-ylamino]-N-(2-oxo-2-phenyl-ethyl)-propionamide;

(S)-N-[1-(5-methoxy-pyridin-2-yl)-cyclohexylmethyl]-2-methyl-2-[4-(4-nitro-phenyl)-oxazol-2-ylamino]-3-phenyl-propionamide;

(S)-2-[4-(4-cyano-phenyl)-oxazol-2-ylamino]-3-(1H-indol-3-yl)-N-[1-(5-methoxy-pyridin-2-yl)-cyclohexylmethyl]-2-methyl-propionamide;

(S)-3-(1H-indol-3-yl)-N-[1-(5-methoxy-pyridin-2-yl)-cyclohexylmethyl]-2-methyl-2-(4-phenyl-oxazol-2-ylamino)-propionamide;

(S)-2-(4-ethyl-oxazol-2-ylamino)-3-(1H-indol-3-yl)-N-[1-(5-methoxy-pyridin-2-yl)-cyclohexylmethyl]-2-methyl-propionamide;

(S)-3-(1H-indol-3-yl)-N-[1-(5-methoxy-pyridin-2-yl)-cyclohexylmethyl]-2-methyl-2-[4-(4-nitro-phenyl)-thiazol-2-ylamino]-propionamide;

(S)-2-(benzooxazol-2-ylamino)-3-(1H-indol-3-yl)-2-methyl-N-(1-pyridin-2-yl-cyclohexylmethyl)-propionamide;

(S)-3-(1H-indol-3-yl)-2-methyl-2-(pyridin-4-ylamino)-N-(1-pyridin-2-yl-cyclohexylmethyl)-propionamide;

(S)-3-(1H-indol-3-yl)-2-(isoquinol-4-ylamino)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(pyrimidin-5-ylamino)-propionamide;

(S)-2-(biphenyl-2-ylamino)-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-m-tolylamino-propionamide;

(S)-3-(1H-indol-3-yl)-2-methyl-2-(6-phenyl-pyridin-2-ylamino)-N-(1-pyridin-2-yl-cyclohexylmethyl)-propionamide;

(R)-3-phenyl-2-phenylamino-N-(1-pyridin-2-yl-cyclohexylmethyl)-propionamide;

(S)-3-(1H-indol-3-yl)-2-methyl-2-phenylethylamino-N-(1-pyridin-2-yl-cyclohexylmethyl)-propionamide;

(S)-2-[(benzofuran-2-ylmethyl)-amino]-3-(1H-indol-3-yl)-2-methyl-N-(1-pyridin-2-yl-cyclohexylmethyl)-propionamide, and

(S)-3-(1H-indol-3-yl)-2-methyl-2-(4-nitro-benzylamino)-N-(1-pyridin-2-yl-cyclohexylmethyl)-propionamide.

General Process for the Preparation of Compounds

One method for making a compound of the formula (I) defined above in which r is 1, j is 0, q is 1, k is 0 and X is -oxazol-2-yl- comprises:

(a) converting a methyl ester of the formula (III)

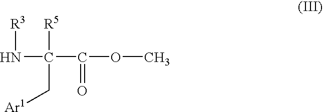

where R³, R⁵ and Ar¹ have the meanings given above via the corresponding p-nitrophenylcarbamate to a urea of the formula (IV):

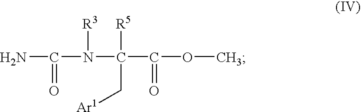

(b) cyclising the urea by reaction with a compound of the formula ArCOCH₂Hal wherein Ar has the meaning given above and Hal represents a halogen to give a compound of the formula (V)

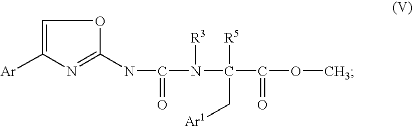

(c) forming an amide bond between the carboxyl group of the compound of formula (V) and an amine of the formula (VI) by removing the methoxy group from the compound of formula (V) and reacting the resulting acid in the presence of O-benzotriazol-1-yl-N,N,N′,N′-tetramethyluronium hexafluorophosphate with the amine of the formula (VI)

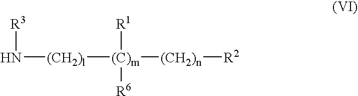

wherein R¹, R², R⁴ and R⁶ are as defined above to give the compound of formula (I) and

(d) optionally converting said compound to a pharmaceutically acceptable salt.

Another method for making a compound of formula (I) as defined above in which k is 0 comprises:

(a) substituting the halogen of a compound of the formula (Ar)ᵣ—(CH₂)ⱼ—(X)_{q}—Hal in which r, j, q, Ar and X are as defined above and Hal represents a halogen atom by an amino group of a compound of the formula (VII) by reaction in the presence of a base with a copper salt as catalyst

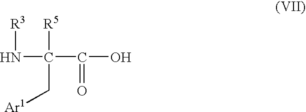

the groups R³, R⁵ and Ar¹ being as defined above;

(b) forming an amide linkage by reacting the resulting acid in the presence of O-benzotriazol-1-yl-N,N,N′,N′-tetramethyluronium hexafluorophosphate with an amine of the formula (VI) as defined above to give the compound of formula (I); and

(c) optionally converting said compound to an acid addition salt.

A further method for making a compound of the formula (I) defined above in which k is 1, which comprises:

(a) protecting with a protective group the amine group of a compound of formula (VII) as defined above;

(b) forming an amide linkage by reacting the protected acid in the presence of O-benzotriazol-1-yl-N,N,N′N′-tetramethyluronium hexafluorophosphate with an amine of the formula (VI) as defined above;

(c) deprotecting the amino group of the resulting amide;

(d) reacting the aldehyde of a compound of the formula (Ar)ᵣ—(CH2)ⱼ—(X)_{q}—CHO in which r, j, q, Ar and X are as defined above by an amino group of the deprotected amide via a reductive amination reaction to give the compound of formula (I); and

(e) optionally converting said compound to an acid addition salt

Pharmaceutical Compositions

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances which may also act as diluents, flavoring agents, solubilizers, lubricants, suspending agents, binders, or tablet disintegrating agents; it can also be an encapsulating material. In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain 5% to about 70% of the active component. Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid form preparations include solutions, suspensions, and emulsions. Sterile water or water-propylene glycol solutions of the active compounds may be mentioned as an example of liquid preparations suitable for parenteral administration. Liquid preparations can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Preferably the pharmaceutical preparation is in unit dosage form. In such form, the preparation is divided into unit doses containing appropriate quantities of the active component. The unit dosage form can be a packaged preparation, the package containing discrete quantities of the preparation, for example, packeted tablets, capsules, and powders in vials or ampoules. The unit dosage form can also be a capsule, cachet, or tablet itself, or it can be the appropriate number of any of these packaged forms.

For preparing suppository preparations, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

The dosage can range from about 0.1 mmol/kg of active compound per kg of bodyweight to about 500 mmol/kg bodyweight. A preferred dosage is about 5 to about 50 mmol of active compound per kg of bodyweight.

Sexual Dysfunction

Although there is no known direct link between the effects of bombesin receptor ligands and sexual function, the presence of receptors in hypothalamic areas might suggest a neuromodulatory effect on functions controlled at a hypothalamic level, and these could include, among others, feeding and sexual behaviour.

Female sexual dysfunction can be grouped into four classes (Scrip's Complete Guide to Women's Healthcare, p. 194-205, April 2000), which include hypoactive sexual desire disorders, sexual arousal disorders, orgasmic disorders or anorgasmy and sexual pain disorders.

Hypoactive sexual desire disorders can be characterized as persistent or recurrent lack of sexual thoughts/fantasies and lack of receptivity to sexual activity, causing personal distress. Common problems include sexual aversion disorders. Sexual arousal disorders can be characterized as persistent or recurrent inability to achieve or maintain adequate sexual excitement, causing personal distress. Common problems include lack of or diminished vaginal lubrication, decreased clitoral and labial sensation, decreased clitoral and labial engorgement and lack of vaginal smooth muscle relaxation. Orgasmic disorders can be characterized as persistent or recurrent difficulty or delay in attaining orgasm after adequate sexual stimulation and arousal, causing personal distress. Sexual pain disorders can be characterized by dyspareunia, (characterised by recurrent or persistent genital pain associated with sexual intercourse), vaginismus (characterised by recurrent or persistent involuntary spasm of the muscles of the outer third of the vagina which interferes with vaginal penetration, causing personal distress) and other pain disorders (characterised by recurrent or persistent genital pain induced by non coital sexual stimulation).

The compounds of this invention are useful in the treatment of female sexual dysfunction, and this includes female sexual dysfunction associated with hypoactive sexual desire disorders, sexual arousal disorders, orgasmic disorders or anorgasmy, or sexual pain disorders.

The psychogenic component of male sexual dysfunction has been classified by the nomenclature committee of the International Society for Impotence Research (and is illustrated in Sachs B. D., Neuroscience and Biobehavioral Review 24: 541-560, 2000) as generalised type, characterised by a general unresponsiveness or primary lack of sexual arousal, and ageing-related decline in sexual arousability, characterised by generalised inhibition or chronic disorders of sexual intimacy. The inventors believe that there are common mechanisms underlying the pathologies of male and female phychogenic sexual dysfunctions.

The compounds of this invention are useful in the treatment of male sexual dysfunction, especially drug induced sexual dysfunction and psychogenic sexual dysfunction associated with generalised unresponsiveness and ageing-related decline in sexual arousability.

Anxiety, Panic Attacks and Social Phobia

Anxiety is a very commonly observed symptom, for which benzodiazepines are the primary treatment agents. Chlordiazepoxide, diazepam, oxazepam, lorazepam, prazepam and alprazolam are most commonly used for this purpose in the United States. However anxiolytic benzodiazepines may also cause sedation, they have muscle-relaxant, sedative-hypnotic, and amnestic side effects; they also tend to potentiate the effects of alcohol. Some tolerance to their effects may develop, withdrawal after chronic use frequently induces rebound anxiety, and long-term use of benzodiazepines, particularly with escalating doses, can lead to dependence. Therefore there is a need for anxiolytic treatments with a reduced dependence liability.

Recent findings suggest a role of bombesin-like peptides in stress and anxiety (Plamondon H. et al. (1996) Soc. Neurosci. 22: Abstract 181.13): antisense oligonucleotides to mRNA for GRP receptors and NMB receptors were infused i.c.v. in rats over 2 days, resulting in a reduction of bombesin binding site density in the brain, as measured by receptor autoradiography. Rats treated with the antisense oligonucleotides spent significantly more time on the anxiogenic fields of an elevated plus maze, or of a trough-tunnel oval maze, reflecting an anxiolytic effect of treatment, as compared to control animals.

The compounds of the instant invention are useful in the treatment of anxiety, panic attacks and social phobia.

Depression

The compounds of the invention are useful in the treatment of depression. The following publication provides evidences of the role of bombesin receptors in depression: Pinnock R. D., et al., Brain Res., 1994, 653:199

Psychoses

The compounds of the invention are useful in the treatment of psychoses. The following publication provides evidences of the role of bombesin receptors in psychoses: Merali., et al., Eur. J. Pharmacol., 1990, 191:281

Sleeping Disorders

The compounds of the invention are useful in the treatment of sleep disorders. The following publication provides evidences of the role of bombesin receptors in sleeping disorders: Even PC., et al., Physiol behav., 1991; 49(3):439-42

Memory Impairment

The compounds of the invention are useful in the treatment of memory impairment. The following publication provides evidences of the role of bombesin receptors in memory impairment: Rashidy., et al., Brain Research., 1998; 814:127-32

Pulmonary Hypertension

Hurel S. J. et al. (Lancet (1996) 348: 1243) have shown that infusion of a GRP receptor antagonist to a patient suffering from pulmonary hypertension was followed by a decrease in the pulmonary systolic pressure. The compounds of the invention are useful in the treatment of pulmonary hypertension.

Lung Repair and Lung Development Disorders

Several studies have emphasised the role of GRP and the GRP receptor in lung repair after injury and in lung development (Spurzem J. R. et al. (1997) Am. J. Respir. Cell. Mol. Biol. 16: 209-211; Wang D. et al. (1996) Am. J. Respir. Cell. Mol. Biol. 14: 409-416; Spindel E. R., Ibidem 14: 407-408). Also, lung injury, including that induced by smoking, leads to increased levels of pulmonary bombesin-like peptides. Findings by Cutz E. et al. (Pediatrics (1996) 98: 668-72) suggest that maternal smoking potentiates hyperplasia of the pulmonary neuroendocrine cells (as measured by the percentage of airway epithelium immunoreactive for bombesin) in the lungs of infants who die of sudden infant death syndrome (SIDS) and that a dysfunction of these cells may contribute to the pathophysiology of SIDS. The compounds of the instant invention are useful in the treatment of lung repair and lung development disorders.

Cancer Treatment

The invention also relates to a method for treating cancer which comprises administering to a patient or a subject, particularly a mammal, more particularly a human, an effective amount of a compound of Formula (I), optionally conjugated with a cytotoxic agent. The method is particularly useful in cancers where tumour cells have a cell surface bombesin receptor, including certain prostate or pancreatic cancers.

When a directly labelled compound of Formula (I) is used for therapeutic purposes, preferably a halogen substituent of Ar as a radionuclide is used. Preferably halogen radionuclides employed for therapy are β-emitting or α-emitting radio-nuclides. The preferred halogen substituents of Ar for treating cancers include ¹³¹I, ²¹¹At, ⁷⁶Br and ⁷⁷Br, ¹³¹I being particularly preferred. Compounds of Formula (I) where Ar is substituted by a radionuclide halogen can easily be prepared via electrophilic aromatic substitution of a corresponding non-radioactive compound wherein Ar is substituted by a halide or an activating group. Such a halide is preferably Br or I Preferred activating groups include tributyl-tin, trimethylsilyl, t-butyldimethylsilyl, and the like.

Conjugation of a compound of Formula (I) with a cytotoxic agent is especially preferred when, in the compound of Formula (I), R² is hydroxy or amino. In such a case, the compounds of the invention may conveniently be linked to a cytotoxic agent, using a bifunctional moiety like glutaric acid or the like to form a conjugate. Suitable cytotoxic agents include compounds such as doxorubicin, anticancer chemotherapy compounds such as those described in The Merck Index, 12th edition, 1996, p. MISC-10.

The use of a conjugate of a compound of Formula (I) with a radionuclide is also provided by the instant invention; preferred radionuclides used for radiotherapy emit an α or β particle; they include ¹⁸⁸Re, ¹³¹I, ²¹¹At, ²¹²Pb, ²¹²Bi, ⁷⁶Br, ⁷⁷Br, and the like (for examples, The Merck Index, 12th edition, 1996, page MISC-93). Said conjugates may be prepared using conventional methods. For example, radionuclides such as ¹⁸⁸Re can be linked to a compound of Formula (I) using a bifunctional chelating agent such as trisuccin (Safavy A. et al. (1993) Bioconj. Chem. 4: 194-8) according to a process adapted from Safavy A. et al. in Cancer (1997) 80 (Suppl): 2354-9. The conjugate may take the form of a compound that is cleaved to release the cytotoxic agent on entry into the tumour cells. Compounds that are rapidly transformed in vivo to yield the parent compound of the above formulae, e.g. by hydrolysis upon entry into a target cell, are preferred.

A method of the present invention for treating a mammalian tumour includes administering to a mammal a composition including a tumour-inhibiting amount of at least one compound of the present invention. Such a tumour-inhibiting amount is an amount of at least one of the subject compounds which permits sufficient tumour localisation of the compound to diminish tumour growth or size. This dosage can range from about 0.1 mmol/kg body weight to about 500 mmol/kg body weight. A preferred dosage is about 5 to about 50 mmol/kg body weight.

The amount of radioactivity administered can vary depending on the type of radionuclide. However, with this in mind the amount of radioactivity that is administered can vary from about 1 millicurie (mCi) to about 800 mCi. Preferably, about 10 mCi to about 600 mCi is administered. Moreover when considering the dosage, the specific activity of the radioactive compound should be taken into consideration. Such a specific activity is preferably very high, e.g. for ¹²³I-labelled compounds the specific activity should be at least about 1,000 Ci/mM to about 50,000 Ci/mM. More preferably the specific activity for ¹²³I-labelled compounds is, e.g., about 10,000 Ci/mM to about 22,000 Ci/mM.

a) Prostate Cancer

Bombesin specifically induces intracellular calcium mobilisation via GRP receptors in human prostate cancer cells (Aprikian A. G. et al. (1996) J. Mol. Endocrinol 16: 297-306). This suggests that the bombesin family of neuropeptides can play a regulatory role in the biology of prostate cancer. The use of antibodies raised against bombesin inhibited the growth of a prostatic carcinoma cell line (Hoosein N. M., (1993) Cancer Bull. 45:436-441).

The compounds of the instant invention are useful in the diagnosis and treatment of prostate cancer.

b) Pancreatic Cancer

Normal and tumour pancreatic cells contain a specific GRP receptor that is expressed more on malignant pancreatic tissues (Hajri A. et al. (1996) Pancreas 12: 25-35). Bombesin-like peptides may stimulate proliferation of human pancreatic cancer cells (Wang Q. J. et al. Int. J. Cancer (1996) 68: 528-34). As a consequence a bombesin receptor antagonist may be used to treat pancreatic cancers. Furthermore, a radiolabelled bombesin receptor antagonist may be used to treat pancreatic cancers.

The compounds of the instant invention are useful in the treatment of pancreatic cancer.

Hepatic Porphyria

The major clinical manifestation of hepatic porphyrias are neurologic symptoms, including abdominal pain, neuropathy, and mental disturbances. It is believed that the neurologic symptoms are caused by an increase of a few gastrointestinal and neurotransmitter polypeptides, including GRP, in the systemic circulation during the acute phase of the disease (Medenica R. et al. (1997) Cell Mol. Biol. 43: 9-27). Treatment with bombesin receptor antagonists may thus reduce the effects of those polypeptides that bind to bombesin receptors, and alleviate the symptomatology of acute porphyria. The compounds of the instant invention are useful in the treatment of hepatic porphyria.

Gastrointestinal Secretory Disturbances

GRP has proved to be a particularly valuable tool in detecting disturbances of gastric secretory function, including those associated with duodenal ulcer disease and Helicobacter pylori infection (McColl K. E. et al. (1995) Aliment. Pharmacol. Ther. 9: 341-7). As a consequence, a radiolabelled bombesin receptor antagonist may be useful to diagnose these conditions. Other gastrointestinal functions such as gallbladder contraction, pancreatic secretion and gastro-oesophageal motility are subject to regulatory controls by GRP, and a radiolabelled bombesin receptor antagonist may be useful to diagnose these conditions.

The compounds of the instant invention are useful in the treatment of gastro-intestinal-secretory disturbances.

Gastrointestinal Disorders

The bombesin receptor has been implicated in gastric acid secretion and gastrointestinal motility Walsh J. H. Ann. Rev Physiol 1988; 50, 41 and Lebacq-Verheyden A et al., in Handbook of Experimental pharmacology 1990;95 (part II) and references therein). As such it could be implicated in colitis, Crohn's disease and inflammatory bowel disease.

Emesis

Bombesin is present in high concentrations in the skin of frogs. As part of a defence reaction, Amphibia secrete emetic substances when swallowed by a predator.

In mammals, bombesin receptors are widely distributed in the GI tract where they cause changes in gastric motility and secretion. Bombesin receptor antagonists of the invention may decrease retching and vomiting and thus be effective in the treatment of emesis, in particular in patients receiving anticancer agents.

Anorexia

Bombesin causes a decrease of glucose intake in mice. In mice lacking the GRP receptor, bombesin no longer showed this effect (Hampton L. et al, Proc. Natl. Acad. Sci. USA, 95: 3188-92, 1998). Bombesin receptor antagonists used in the present invention may increase feeding behavior, and thus be effective in the treatment of anorexia, such as the anorexia of cancer patients.

Pain

The compounds of the invention are useful in the treatment of pain. The following publication provides evidences of the role of bombesin receptors in pain (Cridland and Henry, Brain Research, 584: 163-168, 1992).

Seasonal Affective Disorders

The compounds of the invention are useful in the treatment of seasonal affective disorders. The following publication provides evidences of the role of bombesin receptors in seasonal affective disorders: McArthur A J., et al., J. Neurosci., 2000; 20(14):5496-502

Feeding Disorders

The compounds of the invention are useful in the treatment of feeding disorders. The following publication provides evidences of the role of bombesin receptors in feeding disorders: Ladenheim EE., et al, 1996, 54:705-711.

Pruritus

The compounds of the invention are useful in the treatment of pruritus. The following publication provides evidences of the role of bombesin receptors in pruritus: Maigret C. et al, Eur. J. Pharmacol., 209: 57-61,1991.

Preparative Methods

Throughout this application the following abbreviations have the meanings listed below:

| | |
|---|---|
| NEt₃ | triethylamine |
| THF | tetrahydrofuran |
| HBTU | O-benzotriazol-1-yl-N,N,N′,N′-tetramethyluronium hexafluoro- |
| | phosphate |
| DIPEA | N,N-diisopropylethylamine |
| DMF | N,N-dimethylformamide |
| TEBA | benzyltriethylammonium chloride |
| BOC₂O | di-tert-butyl dicarbonate |
| TFA | trifluoroacetic acid |
| DMA | N,N-dimethylacetamide |
| EtOAc | ethyl acetate |
| MeOH | methanol |
| Trp | tryptophan |
| Ph | phenyl |
| HPLC | high pressure liquid chromatography |
| NP | normal phase |
| RP | reverse phase |
| DMAP | N,N-dimethyl-4-aminopyridine |
| OAc | acetate |
| OB | oestradiol benzoate |
| Prog | progesterone. |

The production of compounds of the formula (I) in which X is oxazolyl is shown in Scheme 1 which illustrates the synthesis of the compounds of Examples 1 to 4 in four steps via Intermediates 4a or 4b. The steps are:

Formation of the p-nitrophenylcarbamate of the methyl ester (Intermediate 1) and subsequent treatment with aqueous ammonia to give a primary urea (Intermediate 2).

Cyclisation of the primary urea with 2-bromo-1-(4-nitro-phenyl)-ethanone to form an oxazole ring (Intermediate 3).

Hydrolysis of the methyl ester protecting group, to give Intermediates 4a or 4b.

Reaction of Intermediate 4a or 4b with the amine Z2, using HBTU to form an amide linkage, to give the desired compounds.

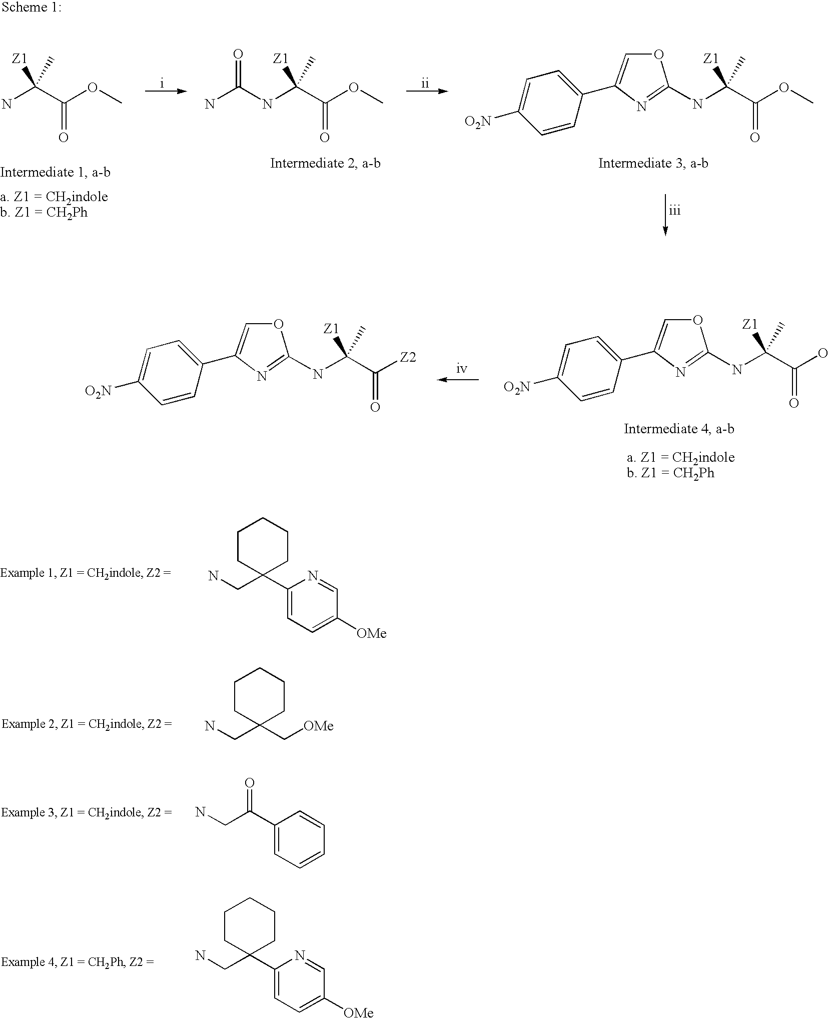

In the above scheme:

i) a) 4-Nitrophenylchloroformate, NEt₃, THF b) NH₃ aq.

ii) 2-bromo-1-(4-nitro-phenyl)-ethanone in either toluene/dioxan at reflux (3a) or 1,2-dichloroethane at reflux (3b)

iii) LiOH, dioxane, H₂O

iv) HBTU, DIPEA, DMF, Z2

Scheme 2 describes the synthesis of the compounds of Examples 5 to 7 from Intermediate 2a:

A primary urea 2a is cyclised with an appropriate bromomethyl ketone containing the group Z3 to form an oxazole ring (Intermediate 5).

Hydrolysis of the methyl ester protecting group of the resulting Intermediate 5a, 5b or 5c gives the Intermediates 6 a-c.

Reaction of an Intermediate 6a, 6b or 6c with [1-(5-methoxy-2-pyridyl)cyclohexyl]methanamine, in the presence of HBTU to form an amide bond, affords the desired compounds.

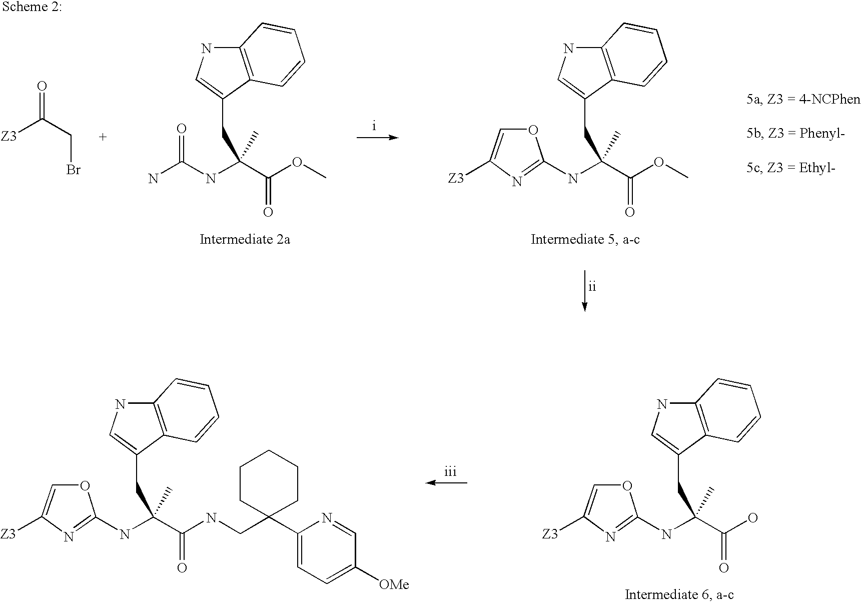

In the above scheme:

i) DMF at 3⁰° C.;

ii) LiOH, dioxane, H₂O;

iii) HBTU, DIPEA,

DMF, [1-(5-methoxy-2-pyridyl)cyclohexyl]methanamine (described in WO 98/07718).

Scheme 3 describes a two step synthesis for the compounds of Examples 8-15. The reactions are preferentially carried out as a “one-pot” process in which:

An aromatic ring of a compound Z5-Br or Z5-CI is appended onto the N-terminal of the illustrated amino acid using a copper catalysed reaction.

Formation of an amide linkage between the resulting acid and [1-(5-methoxy-2-pyridyl)cyclohexyl]methanamine or [1-(2-pyridyl)cyclohexyl]methylamine in the presence of HBTU affords the desired compounds.

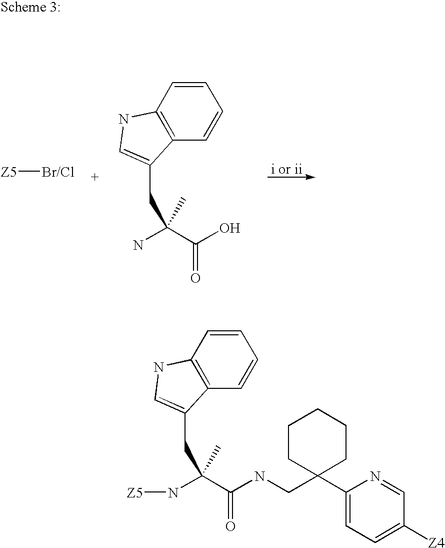

Example 8 Z4=OMe Z5=

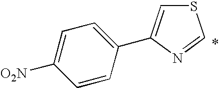

Example 9 Z4=H Z5=

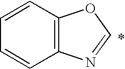

Example 10 Z4=H Z5=

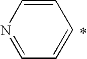

Example 11Z4=H Z5=

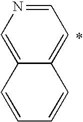

Example 12 Z4=H Z5=

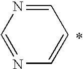

Example 13 Z4=H Z5=

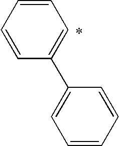

Example 14 Z4=H Z5=

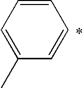

Example 15 Z4=H Z5=

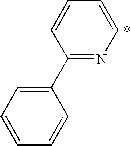

In the above scheme:

i) a) 10% Cul, K₂CO₃, DMF, 130° C.

b) HBTU, DIPEA, DMF, and [1-(5-methoxy-2-pyridyl)cyclohexyl]methanamine (described in WO 98/07718) or [1-(2-pyridyl)cyclohexyl]methylamine (described in WO 98/07718)

ii) a) 5-10% Cul, K₂CO₃, TEBA, Pd(P(o-tolyl)₃)Cl₂, DMF, 130° C.

b) HBTU, DIPEA, DMF, and [1-(5-methoxy-2-pyridyl)cyclohexyl]methanamine (described in WO 98/07718) or [1-(2-pyridyl)cyclohexyl]methylamine (described in WO 98/07718);

* represents the attachment point.

Scheme 4 describes the two step one-pot synthesis of the compound of Example 16:

The aromatic ring is appended onto the N-terminal of the amino acid (Intermediate 8) using a copper catalysed reaction and then an in situ HBTU amide bond formation reaction affords the desired compound.

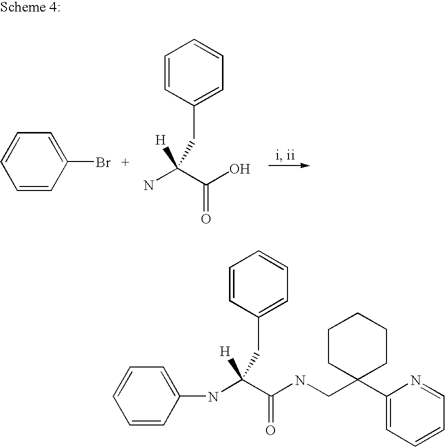

In the above scheme:

i) 10% CuI, K₂CO₃, DMA, 90° C.

ii) HBTU, NEt₃, DMA, [1-(2-pyridyl)cyclohexyl]methylamine (described in WO 98/07718)

Scheme 5 describes the synthesis of the compounds of Examples 17-19 via Intermediate 10 by the steps of:

N-BOC protection of the amino acid (Intermediate 7) which provides the groups R⁵ and Ar¹.

Reaction of the protected amino acid with an amine that provides the groups R¹, R², R⁴ and R⁶ using HBTU to form an amide linkage, and thereby give the Intermediate 9.

N-BOC deprotection of the Intermediate 9 to give Intermediate 10.

Reductive amination of Intermediate 10 with the appropriate aldehyde Z6-CHO to give the desired compounds.

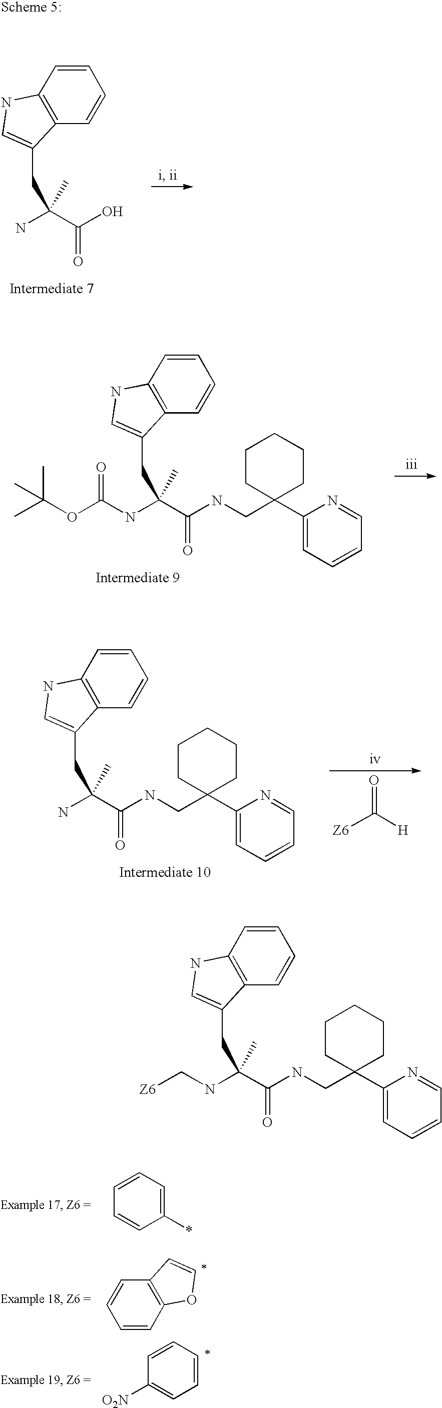

In the above scheme:

i) BOC₂₀, K₂CO₃, dioxane, water

ii) HBTU, DIPEA, [1-(2-pyridyl)cyclohexyl]methylamine (described in WO 98/07718), DMF

iii) TFA, CH₂Cl₂

iv) NaBH(OAc)₃, 1,2-dichloroethane.

represents the attachment point.

Scheme 6 describes the synthesis of Intermediate 13.

The alcohol 11 is methylated using sodium hydride.

The resulting nitrile is reduced using Raney nickel under an atmosphere of hydrogen.

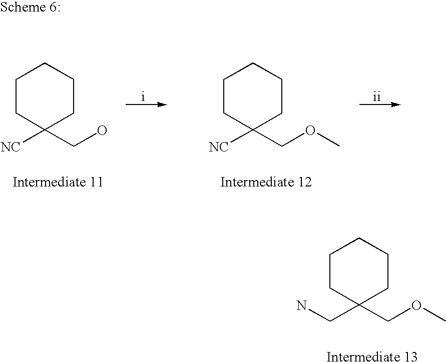

In the above scheme:

i) NaH, CH₃₁, THF

ii) Raney nickel, ethanolic ammonia, H₂, 345 kPa

### Intermediate 13

### C-(1-methoxymethyl-cyclohexyl)-methylamine

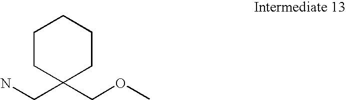

The above compound was prepared as shown in Scheme 6:

1.

Sodium hydride (862 mg, 21.5 mmol, 60% in oil) was taken up in THF (50 ml) under argon at 0° C. To this was added a solution of methyl iodide (1.34 ml, 21.6 mmol) and 1-hydroxy-cyclohexanecarbonitrile (1.0 g, 7.18 mmol; see J. Fröhlich et al., Heterocycles 1994, 37, 1879-91) in THF (30 ml) dropwise over 45 min. Once addition was complete the reaction mixture was stirred at room temperature overnight, and then quenched with i-propanol followed by water (100 ml). The mixture was then extracted with CH₂Cl₂ (2×150 ml). The combined organic phases were dried (MgSO₄) and solvent removed under reduced pressure. Residue was purified by chromatography using heptane/EtOAc (4:1). Removal of solvent under reduced pressure gave 1-methoxymethyl-cyclohexanecarbonitrile (1.1 g, 88%) as a pale yellow oil:

IR (film): 2934, 2861, 2832, 2235, 1476, 1452, 1385, 1211, 1187, 1185, 1126, 1102, 978, 932, 901, 849 cm⁻¹;

¹H NMR (CDCl₃): δ=1.13-1.33 (3H, m), 1.57-1.78 (5H, m), 1.94-2.02 (2H, m), 3.36 (1H, s), 3.42 (3H, s);

2. To the 1-methoxymethyl-cyclohexanecarbonitrile (1.1 g, 7.2 mmol) in ethanolic ammonia (60 ml) was added Raney nickel catalyst (0.55 g, pre-washed with water and ethanol). Reaction mixture was shaken for 16 h under hydrogen (345 kPa) at 30° C. The catalyst was filtered off with extreme caution through a bed of Kieselguhr and washed with ethanol. Removal of the solvent under reduced pressure gave Intermediate 13 (1.12 g, 99%) as a yellow oil.

MS m/e (ES⁺): 158.2 (M⁺+H, 100%);

IR (film): 2926, 2857, 1572, 1452, 1378, 1316, 1190, 1140, 966 cm⁻¹;

¹H NMR (CDCl₃): δ=1.20-1.60 (12H, m), 2.62 (2H, s), 3.23 (2H, s), 3.32 (3H, s)

How the invention may be put into effect will now be further described with reference to the following examples.

### EXAMPLE 1

(S)-3-(1H-Indol-3-yl)-N-[1-(5-methoxy-pyridin-2-yl)-cyclohexylmethyl]-2-methyl-2-[4-(4-nitro-phenyl)-oxazol-2-ylamino]-propionamide (Compound (1))

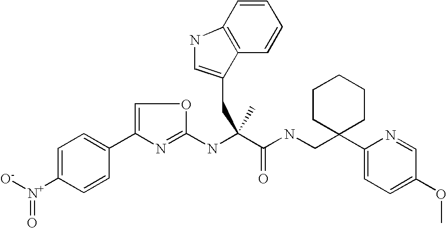

1. To a stirred solution of p-nitrophenylchloroformate (9.27 g, 46 mmol) in THF (200 ml) at 0° C. was added dropwise a solution of H—(S)-αMeTrp-OMe (1a) (10.7 g, 46 mmol) and triethylamine (6.4 ml, 46 mmol) in THF (100 ml) over 1 h. Stirring was continued for a further 30 min at room temperature, after which aqueous ammonia (15 ml) was added. IR after 10 min indicated bands at 1732 and 1660 cm⁻¹. The THF was removed under reduced pressure, and the residue was taken up in EtOAc and washed with 1N HCl (x2), Na₂CO₃ solution (until intense yellow colour subsided, ˜x8), brine, and dried (MgSO₄). The solvent was removed under reduced pressure to give 2a as a foam (10.3 g, 82%):

MS m/e (AP⁺): 276.16 (M⁺+H, 100%);

MS m/e (AP⁻): 274.11 (M—H, 100%);

IR (film): 3383, 1724, 1657, 1600, 1539, 1456, 1374, 1256, 1108, 743 cm⁻¹;

¹H NMR (CDCl₃): δ=1.70 (3H, s), 3.38 (1H, d, J=14.7 Hz), 3.59 (1H, d, J=14.7 Hz), 3.71 (3H, s), 4.22 (2H, s), 5.16 (1H, s), 6.99 (1H, d, J=2.2 Hz), 7.08-7.20 (2H, m), 7.34 (1H, d, J=8.1 Hz), 7.59 (1H, d, J=7.8 Hz), 8.09 (1H, s).

2. The urea (2a) (6.4 g, 23 mmol) and 2-bromo-1-(4-nitro-phenyl)-ethanone (6.0 g, 23 mmol) were stirred in toluene (500 ml)/dioxan (100 ml) and maintained under reflux for 30 h, after which solvent was removed under reduced pressure and the residue was purified by chromatography using a 90g Biotage cartridge. 10% EtOAc in heptane eluted the bromide starting material. 20% EtOAc eluted the desired product. Removal of solvent under reduced pressure gave 3a as a foam (840 mg, 9%):

MS m/e (ES⁺): 420.56 (M+, 100%);

IR (film): 3394, 1732, 1632, 1605, 1574, 1515, 1456, 1334, 1253, 1210, 1108, 1072, 940, 854, 734 cm⁻¹;

¹H NMR (CDCl₃): δ=1.91 (3H, s), 3.46 (1H, d, J=14.6 Hz), 3.69 (3H, s), 3.78 (1H, d, J=14.6 Hz), 5.57 (1H, s), 6.89 (1H, d, J=2.2 Hz), 7.03-7.08 (1H, m), 7.14-7.18 (1H, m), 7.34 (1H, d, J=8.1 Hz), 7.41 (1H, d, J=8.1 Hz), 7.63 (1H, s), 7.85 (2H, d, J=9.0 Hz), 8.05 (1H, s), 8.24 (2H, d, J=8.6 Hz).

3. The ester (3a) (840 mg, 2 mmol) was dissolved in dioxan (50 ml) and LiOH.H₂O (336 mg, 8 mmol) in H₂O (25 ml) was added. The mixture was stirred vigorously overnight, and then neutralised with 1M HCl (8 ml, 8 mmol). The majority of the dioxan was removed under reduced pressure and the product was crystallised, filtered off, washed with water and dried under reduced pressure to give pure 4a (668 mg, 82%):

MS m/e (ES⁺): 407 (M⁺+H);

IR (film): 1633 cm⁻¹;

¹H NMR (DMSO-d₆) δ=1.49 (3H, s), 3.30-3.35 (1H, m, masked by H₂O), 3.59 (1H, d, J=14.7 Hz), 6.86-6.90 (1H, m), 6.99-7.03 (2H, m), 7.30-7.36 (2H, m), 7.48 (1H, s), 7.94 (2H, d, J=9.0 Hz), 8.27-8.30 (3H, m), 10.88 (1H, s), (CO₂H not seen).

4. The acid (4a) (1.148 g, 2.8 mmol), O-benzotriazol-1-yl-N,N,N′N′-tetra-methyluronium hexafluorophosphate (HBTU, 1.06 g, 2.8 mmol), and N,N-diiso-propylethylamine (DIPEA, 490 μl, 2.8 mmol) were stirred in DMF (10 ml) for 5 min before adding DIPEA (490 μl, 2.8 mmol) and [1-(5-methoxy-2-pyridyl)-cyclohexyl]-methanamine (see WO 98/07718, 678 mg, 3.1 mmol). HPLC indicated that reaction was complete within 1 h. Solvent was removed under reduced pressure and the residue was taken up in EtOAc. The organic layer was washed with brine, saturated NaHCO₃ (x3), brine and dried (MgSO₄), after which solvent was removed under reduced pressure. The residue was purified by chromatography using RP silica with 65% MeOH in H₂O. Pure fractions were evaporated to give the desired product as an amorphous solid (1.12 g, 66%):

MPt: 100-105° C.;

MS m/e (ES⁺): 609.63 (M⁺+H, 100%);

IR (film): 3359, 3272, 3054, 2932, 2857, 1628, 1606, 1573, 1515, 1488, 1393, 1336, 1268, 1232, 1181, 1150, 1131, 1097, 1028, 1012, 962, 939, 900, 853, 831, 737 cm⁻¹;

¹H NMR (CDCl₃): δ=1.10-1.60 (8H, m), 1.72 (3H, s), 1.95-2.02 (2H, m), 3.31-3.42 (2H, m), 3.41 (1H, d, J=14.6 Hz), 3.50 (1H, d, J=14.6 Hz), 3.69 (3H, s), 5.34 (1H, s), 6.90-6.97 (2H, m), 7.04-7.09 (2H, m,) 7.14-7.19 (1H, m), 7.33 (1H, d, J=8.1 Hz), 7.46 (1H, d, J=7.8 Hz), 7.54 (1H, s), 7.77 (2H, d, J=8.8 Hz), 8.00 (1H, d, J=2.9 Hz), 8.04 (1H, s), 8.21 (2H, d, J=8.8 Hz); (amide masked by CHCl₃)

HPLC A: Rt. 11.86 min, 99.8/100% purity, 20-100% CH₃CN in H₂O (+0.1% TFA) over 15 min at 1 ml×min⁻¹, Prodigy ODSIII 250×4.6 mm 5 μM, 215 and 254 nm;

HPLC B: Rt. 14.32 min, 100/100% purity, 80:20 MeOH/Tris buffer at pH=9, 1 ml.min⁻¹, Prodigy ODSIII 250×4.6 mm 5 μM, 215 and 254 nm.

### EXAMPLE 2

(S)-3-(1H-Indol-3-yl)-N-(1-methoxymethyl-cyclohexylmethyl)-2-methyl-2-[4-(4-nitro-phenyl)-oxazol-2-ylamino]-propionamide

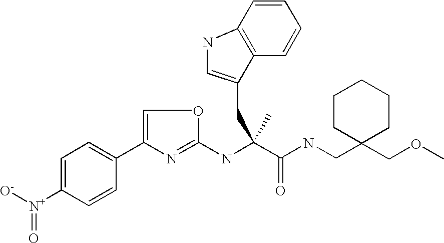

The above compound was synthesized from Intermediate 4a and Intermediate 13 using the same method as used for Example 1. The acid (4a) (203 mg, 0.5 mmol), HBTU (190 mg, 0.5 mmol), and DIPEA (87 μl, 0.5 mmol) were stirred in DMF (10 ml) for 5 min before adding DIPEA (87 μl×2, 1.0 mmol) and Intermediate 13 (94 mg, 0.5 mmol, Scheme 6). After 4 h the solvent was removed under reduced pressure and residue taken up in EtOAc. The organic layer was washed with brine, saturated NaHCO₃ (x3), brine, dried (MgSO₄) and solvent removed under reduced pressure. The residue was heated to 60° C. in MeOH and product filtered off. Drying under reduced pressure gave the desired product as a yellow crystalline solid (214 mg, 78%):

MPt: 189-192° C.;

MS m/e (ES⁺): 546.49 (M⁺+H, 100%);

IR (film): 3285, 2928, 2849, 1637, 1604, 1516, 1453, 1334, 1260, 1108, 1077, 860, 743, 729 cm⁻¹;

¹H NMR (DMSO-d₆): δ=1.10-1.35 (10H, m), 1.44 (3H, s), 2.91-3.01 (3H, m), 3.06-3.12 (1H, m), 3.07 (3H, s), 3.26-3.31 (1H, m), 3.64 (1H, d, J=14.4 Hz), 6.87-6.93 (2H, m), 7.01 (1H, t, J=7.4 Hz), 7.29-7.37 (3H, m), 7.44 (1H, s), 7.94 (2H, d, J=9.0 Hz), 8.26 (2H, d, J=8.8 Hz), 8.34 (1H, s), 10.84 (1H, s);

HPLC A: Rt. 17.07 min, 100/100% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 14.35 min, 100/100% purity, 80:20 MeOH/Tris buffer at pH=9, 1 ml.min⁻¹, Prodigy ODSIII 250×4.6 mm 5 μM, 215 and 254 nm.

### EXAMPLE 3

(S)-3-(1H-Indol-3-yl)-2-methyl-2-[4-(4-nitro-phenyl)-oxazol-2-ylamino]-N-(2-oxo-2-phenyl-ethyl)-propionamide.

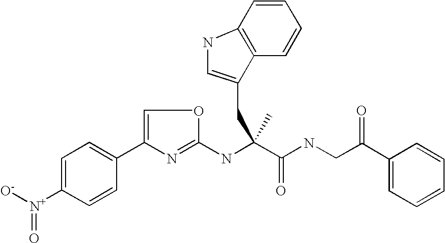

The above compound was synthesised from Intermediate 4a using the same method as used for Example 1. The acid (4a) (203 mg, 0.5 mmol), HBTU (190 mg, 0.5 mmol), and DIPEA (87 μl, 0.5 mmol) were stirred in DMF (10 ml) for 5 min before adding DIPEA (87 μl, 0.5 mmol) and 2-amino-1-phenyl-ethanone (103 mg, 0.6 mmol). After 4 h the solvent was removed under reduced pressure and residue taken up in EtOAc, washed with brine, saturated NaHCO₃ (x3), brine, dried (MgSO₄) and solvent removed under reduced pressure. The residue was purified by chromatography using NP 20g Mega Bond Elut cartridge and 40% EtOAc in heptane as eluent. Evaporation of pure fractions gave the desired product as a yellow amorphous solid (170 mg, 65%):

MPt: 80-90° C.;

MS m/e (AP⁺): 525.83 (16%), 524.44 (M⁺+H, 100%);

IR (film): 3396, 3059, 2983, 2932, 1694, 1628, 1605, 1575, 1514, 1449, 1336, 1284, 1264, 1225, 1181, 1154, 1096, 1072, 1010, 1001, 940, 853, 737 cm⁻¹;

¹H NMR (DMSO-d₆): δ=1.50 (3H, s), 3.39 (1H, d, J=14.7 Hz), 3.64 (1H, d, J=14.6 Hz), 4.53 (1H, d.d, J=18.1 and 5.4 Hz), 4.66 (1H, d.d, J=18.1 and 5.5 Hz), 6.87 (1H, t, J=7.4 Hz), 6.95 (1H, d, J=2.2 Hz), 7.00 (1H, t, J=7.4 Hz), 7.30 (1H, d, J=8.1 Hz), 7.34 (1H, d, J=8.1 Hz), 7.41 (1H, s), 7.50-7.55 (2H, m), 7.62-7.67 (1H,m), 7.94-7.99 (4H, m), 8.24 (1H, t, J=5.4 Hz), 8.27 (2H, d, J=9.0 Hz), 8.31 (1H, s), 10.86 (1H, s);

HPLC A: Rt. 20.83 min, 98.3/99.6% purity, 20-100% CH₃CN in H₂O (+0.1% TFA) over 25 min at 1 ml.min⁻¹, Prodigy ODSIII 250×4.6 mm 5 μM, 215 and 254 nm;

HPLC B: Rt. 6.82 min, 100/100% purity, 80:20 MeOH/Tris buffer at pH=9, 1 ml.min⁻¹, Prodigy ODSIII 250×4.6 mm 5 μM, 215 and 254 nm.

### EXAMPLE 4

(S)-N-[1-(5-Methoxy-pyridin-2-yl)-cyclohexylmethyl]-2-methyl-2-[4-(4-nitro-phenyl)-oxazol-2-ylamino]-3-phenyl-propionamide

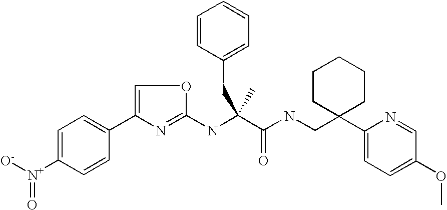

The above compound was synthesised from 1b and 4b using the same methods as used for Example 1. The acid (4b) (120 mg, 0.33 mmol), HBTU (124 mg, 0.33 mmol), and DIPEA (114 μl, 0.66 mmol), and [1-(5-methoxy-2-pyridyl)cyclohexyl]-methanamine (86 mg, 0.4 mmol) were stirred in DMF (4 ml) for 18 h. Solvent removed under reduced pressure and residue taken up in EtOAc. The organic layer was washed with brine, saturated NaHCO₃ (x3), brine, dried (MgSO₄) and solvent removed under reduced pressure. The residue was purified by chromatography using NP silica with 10-80% EtOAc in heptane. Pure fractions were evaporated to give the desired compound as a yellow amorphous solid (90 mg, 49%):

MS m/e (AP⁺): 570.23 (M⁺+H, 100%);

IR (film): 3363, 2930, 2856, 1658, 1651, 1628, 1574, 1515, 1488, 1334, 1268, 1232, 1073, 1030, 938, 852 cm⁻¹;

¹H NMR (DMSO-d₆): δ=0.94-1.46 (11H, m), 1.98-2.10 (2H, m), 3.04-3.14 (2H, m), 3.25-3.32 (1H, m), 3.57 (1H, d, J=13.6 Hz), 3.73 (3H, s), 6.95-7.00 (3H, m), 7.10-7.24 (5H, m), 7.44 (1H, s), 7.93 (2H, d, J=8.8 Hz), 8.14 (1H, d, J=2.8 Hz), 8.27 (2H, d, J=9.2 Hz), 8.36 (1H, s);

HPLC A: Rt. 5.49 min, 99.76% purity, 20-100% CH₃CN in H₂O (+0.1% TFA) over 7 min at 1.5 ml.min⁻¹, Prodigy ODSIII 150×4.6 mm 3 μM at 40° C., 200-300 nm;

HPLC B: Rt. 5.72 min, 99.46% purity, 20-90% CH₃CN/Tris (1 mM) over 7 min at 2 ml.min⁻¹, Prodigy Phenyl-Ethyl, 100×4.6 mm 5 μM at 30° C., 200-300 nm.

### EXAMPLE 5

(S)-2-[4-(4-Cyano-phenyl)-oxazol-2-ylamino]-3-(1H-indol-3-yl)-N-[1-(5-methoxy-pyridin-2-yl)-cyclohexylmethyl]-2-methyl-propionamide

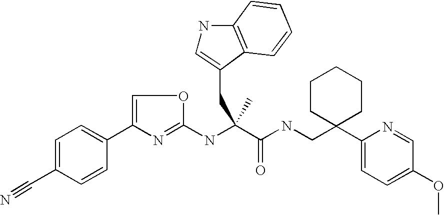

The above compound was synthesised from 2a via 6a as outlined in Scheme 2 using methods analogous to those used for Example 1. The acid (6a) (309 mg, 0.8 mmol), HBTU (303 mg, 0.8 mmol), DIPEA (140 μl, 0.8 mmol) were stirred in DMF (5 ml) for 5 min before adding DIPEA (140 μl, 0.8 mmol) and [1-(5-methoxy-2-pyridyl)cyclohexyl]-methanamine (WO 98/07718) (185 mg, 0.84 mmol). HPLC indicated reaction complete within 1 h. Solvent removed under reduced pressure and residue taken up in EtOAc. Washed with brine, saturated NaHCO₃ (x3), brine, dried (MgSO₄) and solvent removed under reduced pressure. Residue purified by chromatography using RP silica with 65% MeOH in H₂O. Pure fractions were evaporated to give Example 5 as a white amorphous solid (320 mg, 68%):

MPt: 105-108° C.;

MS m/e (ES⁺): 589.32 (M⁺+H, 100%), 590.18 (62%);

IR (film): 3355, 2932, 2857, 2225, 1628, 1572, 1521, 1489, 1456, 1328, 1269, 1232, 1096, 1072, 1029, 938, 844, 741 cm⁻¹;

¹H NMR (CDCl₃): δ=1.20-1.60 (8H, m), 1.70 (3H, s), 1.93-2.03 (2H, m), 3.30-3.52 (4H, m), 3.68 (3H, s), 5.30 (1H, s), 6.89 (1H, d, J=2.4 Hz), 6.94 (1H, d.d, J=8.8 and 2.9 Hz), 7.03-7.09 (2H, m,) 7.14-7.19 (1H, m), 7.20-7.25 (1H, m), 7.33 (1H, d, J=8.1 Hz), 7.46 (1H, d, J=7.8 Hz), 7.50 (1H, s), 7.63 (2H, d, J=8.5 Hz), 7.72 (2H, d, J=8.3 Hz); 8.00 (1H, d, J=2.9 Hz), 8.05 (1H, s);

HPLC A: Rt. 11.63 min, 97.7/100% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 9.20 min, 100/100% purity, 80:20 MeOH/Tris buffer at pH=9, 1 ml.min⁻¹, Prodigy ODSIII 250×4.6 mm 5 μM, 215 and 254 nm.

### EXAMPLE 6

(S)-3-(1H-Indol-3-yl)-N-[1-(5-methoxy-pyridin-2-yl)-cyclohexylmethyl]-2-methyl-2-(4-phenyl-oxazol-2-ylamino)-propionamide

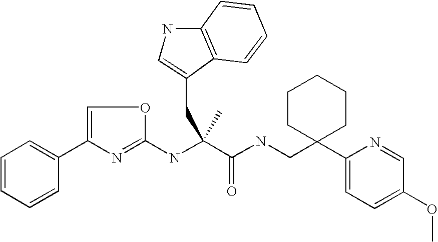

The above compound was synthesised from 2a via 6b as outlined in Scheme 2 using methods-analogous to those used for Example 1. The acid (6b) (57 mg, 0.148 mmol), HBTU (56 mg, 0.148 mmol), DIPEA (26 μl, 0.148 mmol) were stirred in DMF (5 ml) for 5 min before adding DIPEA (26 μl, 0.148 mmol) and [1-(5-methoxy-2-pyridyl)cyclohexyl]-methanamine (see WO 98/07718, 34 mg, 0.148 mmol). HPLC indicated that the reaction was complete within 2 h. Solvent was removed under reduced pressure and the residue was taken up in EtOAc, washed with brine, sat. NaHCO₃ (x3), brine, dried (MgSO₄) and solvent removed under reduced pressure. The residue was purified by chromatography using RP silica with 70% MeOH in H₂O as eluent. Repurification using NP 8g Biotage cartridge with 45% EtOAc in heptane as eluent gave the desired product as a glass (20 mg, 24%):

MPt: 85-90° C.;

MS m/e (ES⁺): 564.06 (M+, 87%), 564.96 (M⁺+H, 100%);

IR (film): 3289, 2931, 2857, 1627, 1569, 1520, 1488, 1456, 1337, 1267, 1233, 1072, 1072, 1030, 939, 739 cm⁻¹;

¹H NMR (DMSO-d₆): δ=0.95-1.45 (11H, m), 2.00-2.10 (2H, m), 3.10-3.25 (2H, m), 3.21 (1H, d, J=14.6 Hz), 3.59 (1H, d, J=14.6 Hz), 3.71 (3H, s), 6.84-7.14 (7H, m), 7.24-7.40 (5H, m,), 7.70 (2H, d, J=7.6 Hz), 8.05 (1H, s), 8.15 (1H, d, J=2.9 Hz), 10.82 (1H, s);

HPLC A: Rt. 12.01 min, 96.8/95.3% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 17.27 min, 100/100% purity, 80:20 MeOH/Tris buffer at pH=9, 1 ml.min⁻¹, Prodigy ODSIII 250×4.6 mm 5 μM, 215 and 254 nm.

### EXAMPLE 7

(S)-2-(4-Ethyl-oxazol-2-ylamino)-3-(1H-indol-3-yl)-N-[1-(5-methoxy-pyridin-2-yl)-cyclohexylmethyl]-2-methyl-propionamide

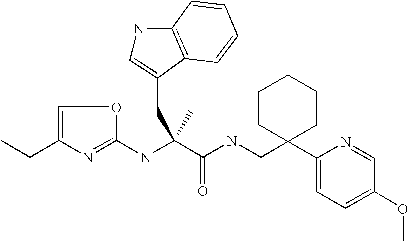

The above compound was synthesised from 2a via 6c as outlined in Scheme 2 using methods analogous to those used for Example 1. The acid (6c) (188 mg, 0.6 mmol), HBTU (228 mg, 0.6 mmol), and DIPEA (105 μl, 0.6 mmol) were stirred in DMF (10 ml) for 5 min before adding DIPEA (105 μl, 0.6 mmol) and [1-(5-methoxy-2-pyridyl)cyclohexyl]-methanamine (see WO 98/07718, 150 mg, 0.65 mmol). HPLC indicated that the reaction was complete within 4 h. Solvent was removed under reduced pressure and residue was taken up in EtOAc, washed with brine, sat. NaHCO₃ (x3), brine, dried (MgSO₄) and solvent removed under reduced pressure. The residue was purified by chromatography using RP silica with 65% MeOH in H₂O. The product was repurified using 20 g Mega Bond Elut silica cartridge with 45% EtOAc in heptane as eluent. Pure fractions were evaporated to give the above compound as a glass (30 mg, 10%):

MPt: 60-65° C.;

MS m/e (ES⁺): 516.24 (M⁺+H, 47%), 517.01 (100%), 538.10 (M⁺+Na, 25

IR (film): 3272, 3054, 2931, 2856, 1651, 1622, 1596, 1573, 1520, 1489, 1457, 1358, 1268, 1232, 1206, 1131, 1083, 1028, 949, 830, 740 cm⁻¹;

¹H NMR (DMSO-d₆): δ=1.10-1.50 (8H, m), 1.11 (3H, t, J=7.4 Hz), 1.29 (3H, s), 2.05-2.15 (2H, m), 2.28-2.34 (2H, m), 3.08-3.18 (3H, m), 3.48 (1H, d, J=14.4 Hz), 3.79 (3H, s), 6.80-6.90 (3H, m), 6.97-7.04 (2H, m,), 7.10-7.20 (3H, m), 7.27-7.30 (2H, m), 8.17 (1H, d, J=2.9 Hz), 10.80 (1H, s);

LCMS: Rt. 1.36 min, 100% purity, 5-100% CH₃CN in H₂O (+0.1% formic acid) over 2 min at 4 ml.min⁻¹, Prodigy ODSIII 50×4.6 mm 5 μM, 215 nm, MS m/e (ES⁺) 515.95 (100%);

HPLC B: Rt. 12.29 min, 100/100% purity, 80:20 MeOH/Tris buffer at pH=9, 1 ml.min⁻¹, Prodigy ODSIII 250×4.6 mm 5 μM, 215 and 254 nm.

### EXAMPLE 8

(S)-3-(1H-Indol-3-yl)-N-[1-(5-methoxy-pyridin-2-yl)-cyclohexylmethyl]-2-methyl-2-[4-(4-nitro-phenyl)-thiazol-2-ylamino]-propionamide

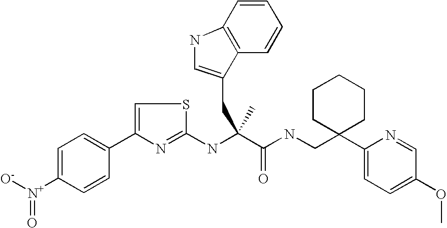

The above compound was synthesised using a one-pot procedure as outlined in Scheme 3. A suspension of H—S-αMeTrp-OH (Intermediate 7) (437 mg, 2 mmol), 2-chloro-4-(4-nitro-phenyl)-thiazole (see Peet, Norton P.; Sunder, Shyam. Reinvestigation of the reported preparation of 3-(4-nitrophenyl)-thiazolo[2,3-c][1,2,4]triazepines, J. Heterocycl. Chem. (1986), 23(2), 593-5; 481 mg, 2 mmol), copper (I) iodide (38 mg, 0.2 mmol), and K₂CO₃ (415 mg, 3 mmol) in DMF (12 ml) under nitrogen was heated to 130° C. for 12 h. The reaction mixture was cooled to ambient temperature before adding HBTU (759 mg, 2 mmol) and [1-(5-methoxy-2-pyridyl)cyclohexyl]-methanamine (see WO 98/07718; 441 mg, 2 mmol). The mixture was stirred overnight, then concentrated in vacuo, after which the residue was partitioned between water (20 ml) and CH₂Cl₂ (30 ml). The organic phase was separated and filtered through silica (3×12 cm) using 500 ml of CH₂Cl₂ and then 500 ml of CH₂Cl₂-ether (1:1). Fractions containing product were concentrated under reduced pressure. The residue was absorbed onto 3.5 g silica and purified by chromatography (3×11 cm) using heptane-EtOAc (1:1.1). The product was repurified using RP chromatography (Biotage KP-C18-HS Flash 12M, 15 ml.min⁻¹, 60-100% MeOH in water). Concentration under reduced pressure gave the desired compound as a pale yellow amorphous solid (27 mg, 2%):

MPt: 110-114° C.;

MS m/e (AP⁺): 624.88 (M+, 100%), 625.70 (M⁺+H, 52%);

IR (film): 3385, 3279, 2931, 2855, 1654, 1595, 1542, 1509, 1456, 1341, 1268, 1231, 1108, 1058, 908, 844, 731 cm⁻¹;

¹H NMR (CDCl₃): δ=1.15-1.55 (8H, m), 1.71 (3H, s), 1.90-2.00 (2H, m), 3.16-3.42 (2H, m), 3.46 (1H, d, J=14.9 Hz), 3.60 (1H, d, J=14.6 Hz), 3.70 (3H, s), 5.51 (1H, s), 6.89-6.93 (3H, m), 6.98 (1H, d, J=8.8 Hz), 7.05-7.10 (1H, m), 7.15-7.25 (2H, m), 7.34 (1H, d, J=8.3 Hz), 7.47 (1H, d, J=7.8 Hz), 7.90 (2H, d, J=9.0 Hz), 7.98 (1H, d, J=2.9 Hz), 9.05 (1H, s), 8.21 (2H, d, J=8.8 Hz);

HPLC A: Rt. 12.30 min, 99.4% purity, 20-100% CH₃CN in H₂O (+0.1% TFA) over 15 min at 1 ml.min⁻¹, Prodigy ODSIII 250×4.6 mm 5 μM, 200-300 nm;

HPLC B: Rt. 15.38 min, 99.5% purity, 80:20 MeOH/Tris buffer at pH=9, 1-ml.min⁻¹, Prodigy ODSIII 250×4.6 mm 5 μM, 200-300 nm.

### EXAMPLE 9

(S)-2-(Benzooxazol-2-ylamino)-3-(1H-indol-3-yl)-2-m thyl-N-(1-pyridin-2-yl-cycloh xylmethyl)-propionamide

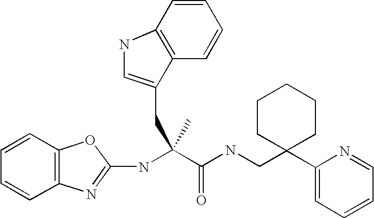

1. The following reagents were combined in the order that they are listed: Intermediate 7 (545 mg, 2.5 mmol), 2-chlorobenzoxazole (384 mg, 2.5 mmol), potassium carbonate (346 mg, 2.5 mmol), benzyltriethylammonium chloride (TEBA, 114 mg, 0.5 mmol), triethylamine (1.04 ml, 7.5 mmol), DMF (12.5 ml), deoxygenated water (1.25 ml), copper (I) iodide (24 mg, 0.125 mmol), trans-dichlorobis(tri-o-tolyl-phosphine)palladium(II) (99 mg, 0.125 mmol). After heating at 100° C. under nitrogen for 24 h the DMF was removed under reduced pressure. The residue was taken up in EtOAc/water and the aqueous phase was acidified to pH 6-6.5 using citric acid. The aqueous phase was extracted with three further portions of EtOAc. The combined organic layers were dried (MgSO₄) and solvent was removed under reduced pressure. The residue was purified by chromatography using 10g NP silica with 0-100% EtOAc in heptane. Crystallisation from CH₂Cl₂ gave (S)-2-(benzoxazol-2-ylamino)-3-(1H-indol-3-yl)-2-methyl-propionic acid (245 mg, 29%). MS m/e (ES⁺) 335.97 (M⁺+H, 100%), 336.69 (85%).

2. The propionic acid (234 mg, 0.7 mmol), HBTU (265 mg, 0.7 mmol), and DIPEA (122 μl, 0.7 mmol) were stirred in DMF (10 ml) for 5 min before adding DIPEA (122 μl, 0.7 mmol) and [1-(2-pyridyl)cyclohexyl]methylamine (WO 98/07718; 140 mg, 0.74 mmol). After 4 h at ambient temperature the solvent was removed under reduced pressure. The residue was purified by chromatography using NP silica with 50% EtOAc in heptane as eluent. Pure fractions were evaporated to give the desired compound as fine needles (44 mg, 3%):

MPt: 198-200° C.;

MS m/e (ES⁺): 508.59 (100%, M⁺+H), 509.92 (10%);

IR (film): 3381, 3222, 3048, 2929, 2856, 1635, 1581, 1552, 1519, 1458, 1353, 1241, 1096, 742 cm⁻¹;

¹H NMR (CDCl₃): δ=1.20-1.60 (8H, m), 1.76 (3H, s), 1.95-2.05 (2H, m), 3.34 (1H, d.d, J=13.2 and 4.9 Hz), 3.45 (1H, d.d, J=13.2 and 5.6 Hz), 3.50 (2H, s), 5.67 (1H, s), 6.78-6.82 (1H, m), 6.89 (1H, d, J=2.2 Hz), 6.99-7.35 (10H, m), 7.43 (1H, d, J=8.1 Hz), 8.01 (1H, s), 8.24 (1H, d, J=4.6 Hz);

HPLC A: Rt. 10.54 min, 100/100% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 10.67 min, 100/100% purity, 80:20 MeOH/Tris buffer at pH=9, 1 ml.min⁻¹, Prodigy ODSIII 250×4.6 mm 5 μM, 215 and 254 nm

### EXAMPLE 10

(S)-3-(1H-Indol-3-yl)-2-methyl-2-(pyridin-4-ylamino)-N-(1-pyridin-2-yl-cyclohexylmethyl)-propionamide

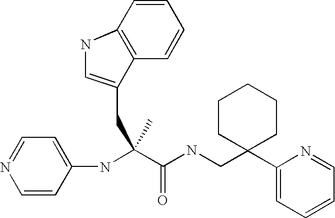

The above compound was prepared on the same scale and using an analogous method as used for Example 9: 1. The method of Example 9 was repeated except that 4-bromopyridine hydrochloride (486 mg, 2.5 mmol) was used.

2. The acid from step 1 (30 mg, 0.1 mmol), HBTU (38 mg, 0.1 mmol), and DIPEA (18 μl, 0.1 mmol) were stirred in DMF (10 ml) for 5 min before adding DIPEA (18 μl, 0.1 mmol) and [1-(2-pyridyl)cyclohexyl]methylamine (WO 98/07718; 19 mg, 0.1 mmol). After 2 h at ambient temperature the solvent was removed under reduced pressure. The residue was taken up in EtOAc and washed with NaHCO₃ solution (x2), brine, and dried (MgSO₄). The solvent was removed under reduced pressure.

The crude product was purified by chromatography using 10 g ISCO Redisep cartridge with EtOAc as eluent. Repurification using 20 g RP-C18 with 70% MeOH in water and subsequent evaporation gave the desired product in crystalline form (6 mg, 13%):

MPt: 180-195° C.;

MS m/e (AP⁺): 468.12 (M⁺+H, 100%), 469.59 (M⁺+2H, 20%);

MS m/e (AP⁻): 467.56 (M⁻, 45%), 466.60 (M⁻−H, 100%), 465.64 (M⁻−2H, 88

IR (film): 3316, 2930, 1651, 1602, 1515, 1430, 1106, 997, 816, 741 cm⁻¹;

NMR (CDCl₃):δ=1.25-1.70 (8H, m), 1.46 (3H, s), 2.00-2.10 (2H, m), 3.27 (1H, d, J=14.9 Hz), 3.30-3.48 (2H, m), 3.36 (1H, d, J=14.9 Hz), 4.43 (1H, s), 6.22 (2H, d, J=5.6 Hz), 6.85 (1H, d, J=2.0 Hz), 6.89-6.93 (1H, m), 7.11-7.37 (5H, m), 7.46-7.54 (2H, m), 8.08-8.13 (4H, m);

HPLC A: Rt. 7.21 min, 96.1/96.5% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 6.02 min, 99.1/100% purity, 80:20 MeOH/Tris buffer at pH=9, 1 ml.min⁻¹, Prodigy ODSIII 250×4.6 mm 5 μM, 215 and 254 nm.

### EXAMPLE 11

(S)-3-(1H-Indol-3-yl)-2-(isoquinolin-4-ylamino)-2-methyl-N-(1-pyridin-2-yl-cyclohexylmethyl)-propionamide

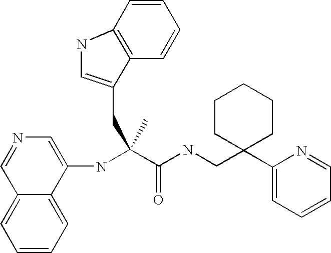

Example 11 was prepared on the same scale and using an analogous method as used for Example 9:

1. The method of Example 9 was followed except that 4-bromoisoquinoline (520 mg, 2.5 mmol) was used.

2. The acid from step 1 (40 mg, 0.12 mmol), HBTU (46 mg, 0.12 mmol), and DIPEA (21 μl, 0.12 mmol) were stirred in DMF (10 ml) for 5 min before adding DIPEA (21 μl, 0.12 mmol) and [1-(2-pyridyl)cyclohexyl]methylamine (WO 98/07718; 23 mg, 0.12 mmol). After 2 h at room temperature the solvent was removed under reduced pressure. The residue was taken up in EtOAc and washed with NaHCO₃ solution (x2) and brine and dried (MgSO₄). The solvent was removed under reduced pressure. The crude product was purified by chromatography using 10 g ISCO Redisep cartridge with 80% EtOAc in heptane as eluent. Repurification using 20 g RP-C18 with 70% MeOH in water and subsequent evaporation gave the desired product as a glass (9 mg, 14%):

MPt: 98-101° C.;

MS m/e (AP⁺): 518.28 (100%, M⁺+H), 517.40 (M⁺, 50%);

MS m/e (AP⁻): 516.53 (75%, M⁻), 515.63 (100%, M⁻−H);

IR (film): 3385, 3278, 3052, 2927, 2849, 1651, 1585, 1520, 1455, 1403, 1343, 781, 740 cm⁻¹;

NMR (CDCl₃): δ=1.20-1.65 (11H, m), 1.93-2.10 (2H, m), 3.35 (1H, d, J=14.6 Hz), 3.39-3.52 (2H, m), 3.48 (1H, d, J=14.9 Hz), 4.62 (1H, s), 6.55-6.59 (1H, m), 6.90 (1H, d, J=2.0 Hz), 7.00 (1H, d, J=8.1 Hz), 7.17-7.28 (4H, m), 7.37-7.55 (4H, m), 7.62 (1H, s), 7.70 (1H, d, J=7.6 Hz), 7.74-7.76 (1H, m), 7.87 (1H, d, J=8.1 Hz), 8.15 (1H, s), 8.63 (1H, s) HPLC A: Rt. 7.52 min, 100/100% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 8.33 min, 99.7/100% purity, 80:20 MeOH/Tris buffer at pH=9, 1 ml.min⁻¹, Prodigy ODSIII 250×4.6 mm 5 μM, 215 and 254 nm

### EXAMPLE 12

(S)-3-(1H-Indol-3-yl)-2-methyl-N-(1-pyridin-2-yl-cyclohexylmethyl)-2-(pyrimidin-5-ylamino)-propionamide

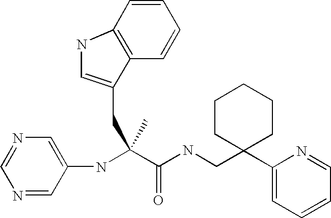

The above compound was prepared on the same scale and using an analogous method as used for Example 9:

1. The method of Example 9 was followed except that 5-bromopyrimidine (397 mg, 2.5 mmol) was used.

2. The acid from step 1 (150 mg, 0.5 mmol), HBTU (190 mg, 0.5 mmol), and DIPEA (87 μl, 0.5 mmol) were stirred in DMF (10 ml) for 5 min before adding DIPEA (87 μl, 0.5 mmol) and [1-(2-pyridyl)cyclohexyl]methylamine (WO 98/07718; 95 mg, 0.5 mmol). After 2 h at room temperature the solvent was removed under reduced pressure. The residue was taken up in EtOAc and washed with NaHCO₃ solution (x2) and brine and dried (MgSO₄). The solvent was removed under reduced pressure. The crude product was purified by chromatography using 10 g ISCO Redisep cartridge with 90% EtOAc in heptane as eluent. Removal of the solvent under reduced pressure gave the desired product as a foam (135 mg, 58%):

MPt: 95-98° C.;

MS m/e (AP⁺): 470.60 (25%), 469.58 (M⁺+H, 100%), 468.77 (M+, 92%);

MS m/e (AP⁻): 467.60 (M⁻−H, 70%), 466.85 (100%);

IR (film): 3291, 3052, 2931, 2857, 1651, 1575, 1519, 1470, 1455, 1427, 1357, 1306, 1265, 1237, 1194, 1156, 1106, 1010, 848, 788, 739 cm⁻¹;

NMR (CDCl₃): δ=1.20-1.65 (8H, m), 1.48 (3H, s), 2.00-2.10 (2H, m), 3.24-3.48 (4H, m), 4.14 (1H, s), 6.88-6.92 (2H, m), 7.13-7.24 (3H, m), 7.37 (1H, d, J=8.1 Hz), 7.48-7.55 (3H, m), 7.86 (2H, s), 8.08-8.10 (1H, m), 8.16 (1H, s), 8.57 (1H, s);

HPLC A: Rt. 8.94 min, 99.3/99.4% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 5.76 min, 95.1/98.7% purity, 80:20 MeOH/Tris buffer at pH=9, 1 ml.min⁻¹, Prodigy ODSIII 250×4.6 mm 5 μM, 215 and 254 nm.

### EXAMPLE 13

(S)-2-(Biphenyl-2-ylamino)-3-(1H-indol-3-yl)-2-methyl-N-(1-pyridin-2-yl-cyclohexylmethyl)-propionamide

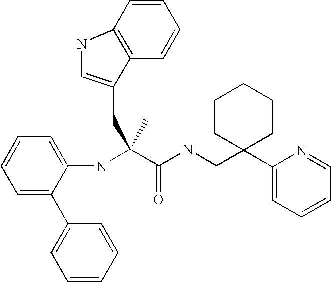

The above compound was prepared on the same scale and using an analogous method as used for Example 9:

1. The method of Example 9 except for the use of 2-bromo biphenyl (583 mg, 2.5 mmol).

2. The acid from step 1 (350 mg, 0.95 mmol), HBTU (400 mg, 1 mmol), NEt₃ (0.5 ml, 3.5 mmol), and 1-(2-pyridyl)cyclohexyl]methylamine (WO 98/07718; 200 mg, 1 mmol) were stirred in DMF (15 ml). After 1 h at room temperature the reaction mixture was diluted with EtOAc (100 ml), washed with NaHCO₃ solution (x2) and dried (MgSO₄). The solvent was removed under reduced pressure. The crude product was purified by chromatography using 0-50% EtOAc in heptane and then 0-30% CH₂Cl₂ in ether as eluent. Removal of the solvent under reduced pressure gave the desired product as a foam (98 mg, 19% for step 2):

MS m/e (AP⁺): 565 (M⁺+Na, 100%), 564 (80%), 542 (M⁺, 30%)

IR (KBr disc): 3404, 2928, 2855, 1650, 1584, 1508, 1489, 1458, 1432 cm⁻¹;

NMR (DMSO-d₆): δ=1.10-1.52 (8H, m), 1.27 (3H, s), 1.95-2.05 (2H, m), 2.95 (1H, d, J=14.4 Hz), 3.02-3.08 (1H, m), 3.08 (1H, d, J=14.6 Hz), 3.28-3.34 (1H, m), −4.36 (1H, s), 6.37 (1H, d, J=8 Hz), 6.49 (1H, d, J=2.2 Hz), 6.71-6.75 (1H, m), 6.82-6.86 (1H, m), 6.95-7.43 (13H, m), 7.52-7.57 (1H, m), 8.33 (1H, d, J=3.7 Hz), 10.81 (1H, s);

HPLC A: Rt. 12.65 min, 99.65% purity, 20-100% CH₃CN in H₂O (+0.1% TFA) over 15 min at 1 ml.min⁻¹, Prodigy ODSIII 250×4.6 mm 5 μM, 200-300 nm;

HPLC B: Rt. 33.05 min, 99.89% purity, 80:20 MeOH/Tris buffer at pH=9, 1 ml.min⁻¹, Prodigy ODSIII 250×4.6 mm 5 μM, 200-300 nm.

### EXAMPLE 14

(S)-3-(1H-Indol-3-yl)-2-methyl-N-(1-pyridin-2-yl-cyclohexylmethyl)-2-m-tolylamino-propionamide

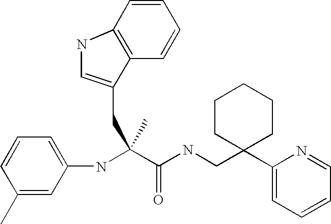

The above compound was prepared using a one-pot procedure analogous to the method used for Example 8. The synthesis was carried out on 1 mmol scale using 1-bromo-3-methyl-benzene (171 mg, 1 mmol). The crude product was purified by chromatography using 25 g NP silica with 25% EtOAc in heptane as eluent. Removal of the solvent under reduced pressure gave the desired compound as a glass (260 mg, 54%):

MPt: 70-75° C.;

MS m/e (AP⁺): 481.33 (100%, M⁺+H), 482.37 (40%);

IR (film): 3385, 3291, 3049, 2929, 2857, 1652, 1607, 1590, 1513, 1456, 1431, 1341, 1302, 1264, 1237, 1177, 1155, 1104, 1010, 774, 741 cm⁻¹;

NMR (DMSO-d₆): δ=1.08-1.50 (8H, m), 1.19 (3H, s), 2.00-2.10 (2H, m), 2.16 (3H, s), 3.03 (1H, d.d, J=12.9 and 5.1 Hz), 3.10 (1H, d, J=14.7 Hz), 3.22 (1H, d, J=14.6 Hz), 3.24-3.30 (1H, m), 5.43 (1H, s), 6.29 (1H, s), 6.30 and 6.44 (each 1H, each d, J=7.6 Hz), 6.87-7.07 (6H, m), 7.15-7.19 (1H, m), 7.29 (1H, d, J=8.0 Hz), 7.33 (1H, d, J=7.8 Hz), 7.48-7.54 (1H, m), 8.31-8.33 (1H, m), 10.81 (1H, s);

HPLC A: Rt. 1-1.04 min, 98.3% purity, 20-100% CH₃CN in H₂O (+0.1% TFA) over 15 min at 1 ml.min⁻¹, Prodigy ODSIII 250×4.6 mm 5 μM, 200-300 nm;

HPLC B: Rt. 16.87 min, 99.5% purity, 80:20 MeOH/Tris buffer at pH=9, 1 ml.min⁻¹, Prodigy ODSIII 250×4.6 mm 5 μM, 200-300 nm.

### EXAMPLE 15

(S)-3-(1H-Indol-3-yl)-2-methyl-2-(6-phenyl-pyridi n-2-ylamino)-N-(1-pyridin-2-yl -cyclohexylmethyl)-propionamide

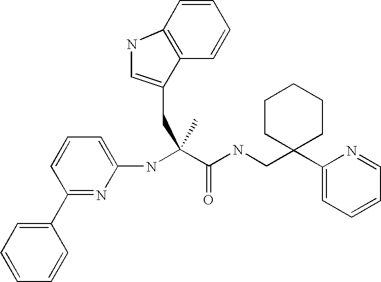

The above compound was prepared using a one-pot procedure analogous to the method used for Example 8. The synthesis was carried out on 0.4 mmol scale using 2-bromo-6-phenyl-pyridine (95 mg, 0.4 mmol). The crude product was purified by chromatography using 25 g NP silica with 55% EtOAc in heptane as eluent. Removal of the solvent under reduced pressure gave the desired product as a foam (260 mg, 54%):

MS m/e (AP⁺) 544.31 (100%, M⁺+H), 545.35 (35%);

MS m/e (AP⁻) 542.29 (100, M⁻−H), 543.31 (M⁻, 40%);

IR (film): 3407, 3276, 3056, 2930, 2857, 1651, 1595, 1576, 1519, 1486, 1467, 1455, 1439, 1339, 1264, 1180, 1157, 1105, 1028, 1009, 991, 804, 763, 739 cm⁻¹;

¹H NMR (CDCl₃) δ=1.03-1.60 (8H, m), 1.53 (3H, s), 1.90-2.03 (2H, m), 3.32-3.45 (3H, m), 3.65 (1H, d, J=14.6 Hz), 4.67 (1H, s), 6.13 (1H, d, J=8.3 Hz), 6.77-7.50 (14H, m), 7.97 (2H, d, J=7.1 Hz), 8.02 (1H, s), 8.23-8.25 (1H, m);

HPLC A: Rt. 4.21 min, 96.8% purity, 20-100% CH₃CN in H₂O (+0.1% TFA) over 7 min at 1.5 ml.min⁻¹, Prodigy ODSIII 150×4.6 mm 5 μM, 200-300 nm.

### EXAMPLE 16

(R)-3-Phenyl-2-phenylamino-N-[1-pyridin-2-yl-cyclohexylmethyl)-propionamide

The above compound was synthesised as a two step process from

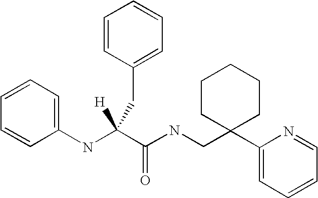

Intermediate 8 as shown in Scheme 4:

1. To a solution of Intermediate 8 (0.5 g, 3 mmol) and bromobenzene (0.35 ml, 3.3 mmol) in DMA (5 ml) under nitrogen was added potassium carbonate (0.6 g, 4.3 mmol) and copper (I) iodide (50 mg, 0.26 mmol) after which the mixture was heated to 90° C. for 1.5 h. Solvent was removed under reduced pressure and the residue was purified by flash chromatography eluting with 5% MeOH in CH₂Cl₂. Removal of solvent under reduced pressure gave (R)-3-phenyl-2-phenylamino-propionic acid as an oil (0.41 g, 56%): MS m/e (AP⁺): 242 (M⁺+H, 100%).

2. The acid from step 1 (0.40 g, 1.66 mmol), HBTU (0.6 g, 1.8 mmol), and NEt₃ (0.5 ml, 3.5 mmol), and 1-(2-pyridyl)cyclohexyl]methylamine (WO 98/07718; 0.35 mg, 1.8 mmol) were stirred in DMF (15 ml). After 1 h at ambient temperature the reaction mixture was diluted with EtOAc (100 ml), washed with NaHCO₃ solution (x2) and dried (MgSO₄). The solvent was removed under reduced pressure. The crude product was purified by chromatography using 50% EtOAc in heptane and then RP C18 silica with 70% MeOH in water as eluent. Removal of the solvent under reduced pressure gave the desired product as a white amorphous solid (0.15 g, 22%):

MPt: 113-115° C.;

MS m/e (AP⁺): 414.22 (M⁺+H, 100%);

IR (KBr disc): 3300, 2931, 2858, 1649, 1605, 1589, 1523, 1498, 1432, 1318, 748 cm⁻¹;

NMR (CDCl₃): δ=1.20-1.70 (8H, m), 1.90-2.15 (2H, m), 2.91 (1H, d.d, J=14.2 and 8.8 Hz), 3.27 (1H, d.d, J=14.2 and 4.4 Hz), 3.38 (1H, d.d, J=13.2 and 5.5 Hz), 3.48 (1H, d.d, J=13.2 and 6.1 Hz), 3.80 (1H, d, J=3.4 Hz), 3.88-3.93 (1H, m), 6.44 (2H, d, J=7.8 Hz), 6.74 (1H, t, J=11.3 Hz), 6.90-7.45 (11H,m), 8.28 (1H, d, J=3.6 Hz);

HPLC A: Rt. 4.51 min, 100% purity, 20-100% CH₃CN in H₂O (+0.1% TFA) over 10 min at 1.5 ml.min⁻¹, Prodigy ODSIII 250×4.6 mm 5 μM, 200-300 nm;

HPLC B: Rt. 13.15 min, 99.14% purity, 80:20 MeOH/Tris buffer at pH=9, 1 ml.min⁻¹, Prodigy ODSIII 250×4.6 mm 5 μM, 200-300 nm;

### EXAMPLE 17

(S)-3-(1H-Indol-3-yl)-2-methyl-2-phenylethylamino-N-(1-pyridin-2-yl-cyclohexylmethyl)-propionamide

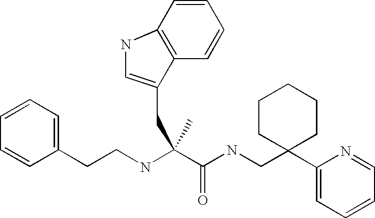

The above compound was prepared as shown in Scheme 5 via Intermediate 10:

1. To a stirred solution of H—(S)-αMe-Trp-OH (7) (10 g, 46 mmol) and di-t-butyl-dicarbonate (10 g, 46 mmol) in dioxan (100 ml) was added water (20 ml) and potassium carbonate (10 g, 74 mmol). After 4 h the reaction mixture was acidified with 2N hydrochloric acid (150 ml) and product was extracted with EtOAc (2×200 ml). The combined organic phases were dried (MgSO₄) and evaporated under reduced pressure. The residue was purified by flash chromatography using EtOAc as eluent. Removal of solvent under reduced pressure gave Boc-(S)-aMeTrp-OH as an orange oil (14.5 g, 99%). To a stirred solution of Boc-(S)-αMeTrp-OH (7 g, 22 mmol) in DMF (100 ml) was added HBTU (8.0 g, 22 mmol), triethylamine (5 ml, 35 mmol), and [1-(2-pyridyl)cyclohexyl]methylamine (WO 98/07718; 4.2 g, 22 mmol). After 1 h the reaction mixture was diluted with EtOAc (300 ml), washed with 2N hydrochloric acid (2×200 ml), dried (MgSO₄) and evaporated under reduced pressure at 60° C. The residue was purified by flash chromatography. Elution with 5% MeOH in CH₂Cl₂ and subsequent removal of solvent under reduced pressure gave 9 as yellow oil (8.3 g, 77%):

MS m/e (AP⁺): 491 (M⁺+H, 100%), 513 (M⁺+Na, 20%);

IR (film): 3339, 2929, 2858, 1704, 1659, 1651, 1589, 1519, 1487, 1366, 1249, 1164, 1070, 908, 737 cm⁻¹;

NMR (CDCl₃): δ=1.20-1.70 (20H, m), 2.00-2.12 (2H, m), 3.25-3.50 (4H, m), 5.05-5.20 (1H, br.s), 6.92 (1H, d, J=2.0 Hz), 7.02-7.32 (6H, m), 7.51 (1H, d, J=8.0 Hz), 7.59-7.64 (1H, m), 8.03 (1H, s), 8.48 (1H, d, J=4 Hz);

2. To a stirred solution of Intermediate 9 (8.2 g, 16.5 mmol) in CH₂Cl₂ (100 ml) was added TFA (3.0 ml, 39 mmol). After 18 h the solvent was removed under reduced pressure at 60° C. The residue was treated cautiously with saturated sodium carbonate solution (200 ml) before extracting with EtOAc (3×200 ml). The combined organic phases were dried (MgSO₄) and evaporated under reduced pressure at 60° C. The residue was purified by flash chromatography. Elution with 0-5% MeOH in CH₂Cl₂ and subsequent removal of solvent under reduced pressure gave Intermediate 10 as white foam (4.85 g, 75%):

MPt: 65-68° C.;

MS m/e (AP+): 391 (M⁺+H, 100%);

IR (KBr disc): 3367, 2926, 2855, 1648, 1589, 1569, 1522, 1455, 1430, 1366, 1341, 1234, 842, 784, 742 cm⁻¹;

NMR (CDCl₃): δ=1.20-1.80 (13H, m), 1.98-2.20 (2H, m), 2.83 (1H, d, J=14.2 Hz), 3.33 (1H, d, J=14.2 Hz), 3.38 (2H, d, J=5.6 Hz), 6.98-7.20 (6H, m), 7.50-7.75 (3H, m), 8.05-8.15 (1H, s), 8.49-8.51 (1H, m);

3. To a stirred solution of Intermediate 10 (293 mg, 0.75 mmol) and phenacetal-dehyde (90 mg, 0.75 mmol) in 1,2-dichloroethane (20 ml) was added solid sodium triacetoxyborohydride (316 mg, 1.5 mmol). After stirring overnight, saturated NaHCO₃ solution was added—effervescence was observed. The aqueous phase was extracted with CH₂Cl₂. The combined organic phases were dried (MgSO₄) and solvent was removed under reduced pressure. The residue was purified by chromatography using 20 g RP-C18 with 0-50% MeOH in water followed by 20 g NP silica with 45% EtOAc in heptane. Removal of solvent under reduced pressure gave the desired compound as a glass (60 mg, 16%):

MS m/e (ES⁺): 496.56 (28%), 495.5 (52%, M⁺+H), 364.43 (22%), 269.34 (51%), 268.90 (88%), 248.37 (100%);

IR (film): 3274, 3058, 2928, 2856, 1651, 1588, 1568, 1519, 1469, 1454, 1431, 1355, 1263, 1236, 1155, 1117, 1053, 1030, 1009, 992, 930, 782, 742 cm⁻¹;

¹H NMR (CDCl₃): δ=1.20-1.65 (11H, m), 2.00-2.20 (2H, m), 2.40-2.75 (4H, m), 2.94 and 3.05 (each 1H, each d, J=14.4 Hz), 3.41 (2H, d, J=6.1 Hz), 6.74 (1H, d, J=2.2 Hz), 7.04-7.25 (9H, m), 7.32 (1H, d, J=7.8 Hz), 7.55-7.60 (3H, m), 7.90 (1H, s), 8.55-8.58 (1H, m);

HPLC A: Rt. 8.52 min, 99.0/98.6% purity, 20-100% CH₃CN in H₂O (+0.1% TFA) over 15 min at 1 ml.min⁻¹, Prodigy ODSIII 250×4.6 mm 5 μM, 215 and 254 nm;

HPLC B: Rt. 23.84 min, 99.6/100% purity, 80:20 MeOH/Tris buffer at pH=9, 1 ml.min⁻¹, Prodigy ODSIII 250×4.6 mm 5 μM, 215 and 254 nm.

### EXAMPLE 18

(S)-2-[(Benzofuran-2-ylmethyl)-amino]-3-(1H-indol-3-yl)-2-methyl-N-(1-pyridin-2-yl-cyclohexylmethyl)-propionamide

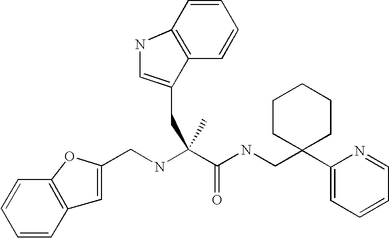

The above compound was prepared as shown in Scheme 5 via Intermediate 10:

To a stirred solution of Intermediate 10 (150 mg, 0.38 mmol) and benzofuran-2-carbaldehyde (56 mg, 0.38 mmol) in 1,2-dichloroethane (5 ml) was added solid sodium triacetoxyborohydride (162 mg, 0.77 mmol). After stirring at room temperature for 48 h saturated NaHCO₃ solution was added—effervescence was observed. The aqueous phase was extracted with EtOAc. The combined organic phases were dried (MgSO₄) and solvent removed under reduced pressure. The residue was purified by chromatography using 60% EtOAc in heptane. Removal of solvent under reduced pressure gave the desired product as an amorphous white solid (29 mg, 15%):

MS m/e (ES⁺): 521.08 (M⁺+H, 100%), 391.06 (50%);

IR (film): 3268, 3056, 2930, 2856, 1656, 1588, 1569, 1519, 1469, 1454, 1431, 1355, 1342, 1255, 1171, 1105, 1052, 1009, 909, 788, 740 cm⁻¹;

¹H NMR (CDCl₃): δ=1.20-2.20 (14H, m), 3.08 (1H, d, J=14.4 Hz), 3.14 (1H, d, J=14.8 Hz), 3.45-3.49 (2H, m), 3.66 (1H, d, J=14.4 Hz), 3.76 (1H, d, J=14.8 Hz), 6.33 (1H, s), 6.84-6.88 (1H, m), 7.00-7.65 (12H, m), 8.32 (1H, s), 8.39 (1H, d, J=4.0 Hz);

HPLC A: Rt. 8.86 min, 99.7/99.1% purity, 20-100% CH₃CN in H₂O (+0.1% TFA) over 15 min at 1 ml.min⁻¹, Prodigy ODSIII 250×4.6 mm 5 μM, 215 and 254 nm.

### EXAMPLE 19

(S)-3-(1H-Indol-3-yl)-2-methyl-2-(4-nitro-benzylamino)-N-(1-pyridin-2-yl-cyclohexylmethyl)-propionamide

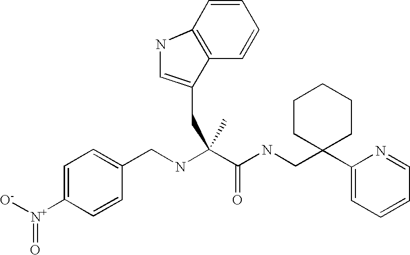

The above compound was prepared as shown in Scheme 5 via Intermediate 10. To a stirred solution of Intermediate 10 (150 mg, 0.38 mmol) and 4-nitrobenzaldehyde (58 mg, 0.38 mmol) in 1,2-dichloroethane (5 ml) was added solid sodium triacetoxyborohydride (114 mg, 0.54 mmol). After stirring at room temperature for 24 h saturated NaHCO₃ solution was added—effervescence was observed. The aqueous phase was extracted with EtOAc. The combined organic phases were dried (MgSO₄) and solvent removed under reduced pressure. The residue was purified by chromatography using 60% EtOAc in heptane. Repurification using RP silica with 45% MeOH in water (+1% acetic acid) gave pure product. The pure fractions were combined, basified (sodium carbonate), and extracted with EtOAc. Removal of solvent under reduced pressure gave the desired compound as a glass (10.5 mg, 5 MPt: 58-60° C.;

MS m/e (ES⁺): 526.15 (M⁺+H, 100%), 527.14 (33%);

IR (film): 3365, 2924, 2856, 1652, 1513, 1429, 1346, 1257, 1048 cm⁻¹;

¹H NMR (DMSO-d₆): δ=1.10-1.55 (8H, m), 1.19 (3H, s), 1.88-2.08 (2H, m), 2.25-2.30 (1H, m), 2.95-3.02 (2H, m), 3.10-3.20 (1H, m), 3.17-3.27 (1H, m), 3.50-3.80 (2H, m), 6.93-7.63 (11H, m), 8.12 (2H, d, J=8.8 Hz), 8.42 (1H, d, J=3.6 Hz), 10.86 (1H, s).

### EXAMPLE 20

### BB₁ and BB₂ Binding Assays

In the following experiments, measurement of BB₁ and BB₂ binding was as follows. CHO-K1 cells stably expressing cloned human NMB (for BB₁ assay) and GRP receptors (for BB₂ assay) were routinely grown in Ham's F12 culture medium supplemented with 10% foetal calf serum and 2 mM glutamine. For binding experiments, cells were harvested by trypsinization, and stored frozen at −70° C. in Ham's F12 culture medium containing 5% DMSO until required. On the day of use, cells were thawed rapidly, diluted with an excess of culture medium, and centrifuged for 5 min at 2000 g. Cells, were resuspended in 50 mM Tris-HCl assay buffer (pH=7.4 at 21° C., containing 0.02% BSA, 40 μg/mL bacitracin, 2 μg/mL chymostatin, 4 μg/mL leupeptin, and 2 μM phosphoramidon), counted, and polytronned (setting 5, 10 s) before centrifuging for 10 min at 28,000 g. The final pellet was resuspended in assay buffer to a final cell concentration of 1.5 □10⁵/mL. For binding assays, 200 μL aliquots of membranes were incubated with [¹²⁵I][Tyr⁴]bombesin (<0.1 nM) in the presence and absence of test compounds (final assay volume 250 μL) for 60 min and 90 min for NMB and GRP receptors, respectively. Nonspecific binding was defined by 1 μM bombesin. Assays were terminated by rapid filtration under vacuum onto Whatman GF/C filters presoaked in 0.2% PEI for >2 h, and washed 50 mM Tris-HCl (pH=6.9 at 21° C.; 6 □1 mL). Radioactivity bound was determined using a gamma counter.

All-competition data was-analysed using nonlinear regression utilising iterative curve-plotting procedures in Prism® (GraphPad Software Inc., San Diego, USA). IC₅₀ values were corrected to Kᵢ values using the Cheng-Prusoff equation (Cheng Y., Prusoff W. H., Biochem. Pharmacol. 22: 3099-3108, 1973). The results obtained are listed in Table 1.

TABLE 1
Human NMB and GRP receptor binding affinities

| Example No. | NMB Kᵢ (nM) | GRP Kᵢ (nM) |
|---|---|---|
| 1 | 4 | 24 |
| 2 | 469 | |
| 3 | 5580 | |
| 4 | 16 | 2820 |
| 5 | 19 | 1385 |
| 6 | 106 | 1190 |
| 7 | 213 | 1770 |
| 8 | 15 | |
| 9 | 2080 | |
| 10 | 303 | |
| 11 | 1249 | |
| 12 | 3163 | |
| 13 | 824 | |
| 14 | 653 | |
| 15 | 3371 | |
| 16 | 137 | |
| 17 | 616 | 2620 |
| 18 | 2400 | |
| 19 | 652 | |

### EXAMPLE 21

Effect of (S)-3-(1H-Indol-3-yl)-N-[1-(5-methoxy-pyridin-2-yl)-cyclohexylmethyl]-2-methyl-2-[4-(4-nitro-phenyl)-oxazol-2-ylamino]-propionamide (Compound (1) in PEG 200 on Female Rat Sexual Proceptivity

Ovariectomised adult female Sprague Dawley rats (180-200 g) were housed in groups of 6 in a reversed lighting system of 12 h light:dark (lights off 7.00-19.00 h). Two weeks after ovariectomy they were used for sexual activity tests. Animals were Adapted to the apparatus (in the absence of stimuli animals) for 10 min on 2 Consecutive days prior to testing. The experiments started at least 5 h into the dark period. Tests were carried out in a circular arena of 90 cm diameter, surrounded by a 30 cm high wall. Two small cages with wire-mesh front (15×15 cm) are fixed into the wall such that the front of the cage is “flush” with the wall and the 2 cages are opposite each other. They contain two stimuli animals: an intact sexually experienced male and a receptive female (ovariectomised, primed with 5 μg oestradiol benzoate dissolved in corn oil and injected subcutaneously 48 h before the test and with 0.5 mg of progesterone 4 h before the test). Sexually naïve test and control animals were used. Forty eight hours before the tests, both the test and control animals were primed with 5 μg oestradiol benzoate. Test animals were treated with the above compound (1)(30-100 mg/kg) which was dissolved in PEG 200 vehicle and administered orally in a 1 ml/kg volume 1 h before each test. For animals used as positive controls, progesterone (0.5 mg/0.1 ml) was dissolved in corn oil and administered subcutaneously (s.c.), 4 h before the test. Test and control animals were introduced one at a time for 10-minute periods into the arena. During the 10-min test, the time that the test or positive control animal spent investigating each stimulus animal was noted. The arena was thoroughly cleaned between animals. The position of the male/female stimuli boxes was randomised between animals, in order to avoid place preference. The difference in the percentage of time spent investigating male minus female was calculated, out of the total time spent investigating stimuli animals.

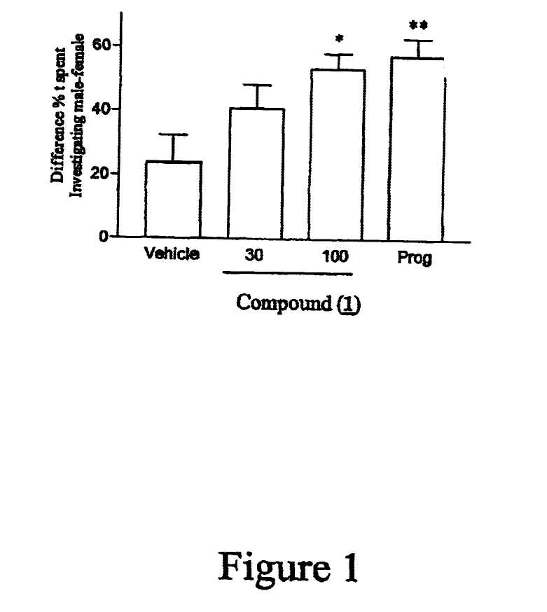

It was found (see FIG. 1) that compound (1) dose-dependently (30-100 mg/kg) increased the difference in the percentage of time spent investigating the male stimuli minus female stimuli, with a MED of 100 mg/kg. The effect of this dose was similar to that of progesterone (maximal). (*P<0.05, **P<0.01 Kruskal-Wallis followed by Mann-Whitney test, vs vehicle).

### EXAMPLE 22

Effect of Compound (1) in Methyl Cellulose on Female Rat Sexual Proceptivity.

Example 21 was repeated except that compound (1) (3-30 mg/kg) was dissolved in 0.5% methyl cellulose and was administered p.o. in a dosing volume of 3 ml/kg 1 h before tests. Progesterone (0.5 mg/0.1 ml) was dissolved in corn oil and administered s.c., 4 h before test, as a positive control.

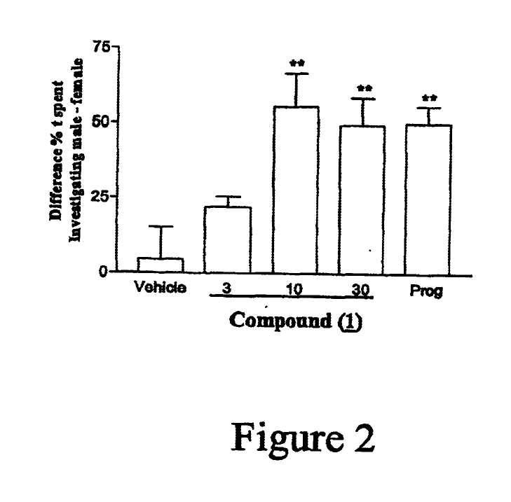

The compound (1) dose-dependently (3-30 mg/kg) increased the difference in the percentage of time spent investigating the male stimuli minus female stimuli, with a MED of 10 mg/kg. This represents a 10-fold increase in potency compared to the oral results obtained in the PEG 200 vehicle (MED=100 mg/kg). The results are shown in FIG. 2 in which bars represent percentage of time spent investigating male, minus the percentage of time spent investigating the female stimuli ±SEM, (n=6-9 per group). *P<0.05, **P<0.01 vs vehicle (one-way ANOVA followed by Dunneft's test vs vehicle group).

### EXAMPLE 23

Effect of Compound (1) in PEG 200 on Female Rat Sexual Receptivity.

Ovariectomised adult female Sprague Dawley rats (180-200 g) were housed in groups of 6 in a reversed lighting system of 12 h light:dark (lights off 7.00-19.00 h). Two weeks after ovariectomy they were used for sexual activity tests. The experiments started at least 5 h into the dark period. Compound (1) was dissolved in PEG 200 vehicle and administered orally. Quinelorane dihydrochloride (LY 163,502, 6.25 μg/kg) was dissolved in water and administered s.c., as a positive control. Both compounds were administered in a 1-ml/kg volume. Forty eight hours before tests, the animals were primed with 5 μg oestradiol benzoate dissolved in corn oil and injected s.c. The females were placed with a series of vigorous male rats and subjected to 10 mounts. The lordotic response of the animal was recorded and expressed as a percentage of the mounts (i.e. lordosis quotient, LQ). Treatment induced LQ=0-10% in most of the animals, which were considered non-receptive (NR). Animals showing higher LQ were not included in the study. Each rat was tested prior to administration of the compound (1) and then tested similarly at 1 h and at 90 min post-injection of compound (1) or quinelorane respectively.

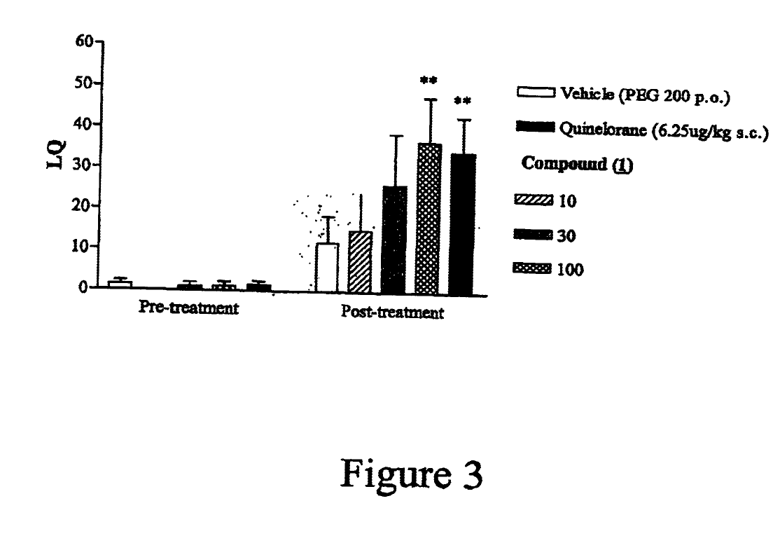

A single administration of quinelorane (6.25 μg/kg) significantly (P<0.01) increased the LQ, 90 min after administration, compared to the LQ shown before administration (paired t test). A single oral administration of compound (1) dose-dependently (10-100 mg/kg) increased the LQ 1 h after administration, with a MED of 100 mg/kg (P<0.01) compared to the LQ shown before administration (paired t test). The effect of compound (1) (100 mg/kg) was similar to the effect of quinelorane (6.25 μg/kg) as is shown in FIG. 3.

1. A compound of formula (I) or a pharmaceutically acceptable salt thereof: 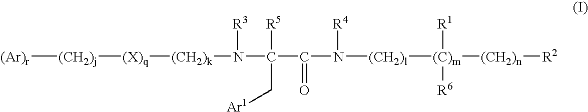
wherein:
j is 0, 1 or 2;
k is 0 or 1;
l is 0, 1, 2, or 3;
m is 0 or 1;
n is 0, 1 or 2;
q is 0 or 1;
r is 0 or 1; when r is 0, Ar is replaced by hydrogen;
Ar is phenyl, pyridyl, pyrimidyl, thienyl, furyl, imidazolyl, pyrrolyl or thiazolyl each unsubstituted or substituted by from 1 to 3 substituents selected from acetyl, alkoxy, alkyl, amino, cyano, halo, hydroxy, nitro, sulfonamido, sulfonyl, —CF₃, —OCF₃, —CO₂H, —CH₂CN, —SO₂CF₃, —CH₂CO₂H and —(CH₂)₅NR⁷R⁸ wherein s is 0, 1, 2 or 3 and R⁷ and R⁸ are each independently selected from H, straight or branched alkyl of up to 6 carbon atoms, or R⁷ and R⁸, together with the nitrogen atom to which they are linked, can form a 5- to 7-membered aliphatic ring which may contain 1 or 2 oxygen atoms;
R¹ is hydrogen, straight or branched alkyl of up to 6 carbon atoms or cycloalkyl of between 5 and 7 carbon atoms which may contain 1 or 2 nitrogen or oxygen atoms;
R⁶ is hydrogen, methyl or forms with R¹ an aliphatic ring of from 3 to 7 atoms which can contain an oxygen or nitrogen atom, or together with R¹ is a carbonyl group;
Ar¹ is independently selected from Ar or is indolyl or pyridyl-N-oxide;
R³, R⁴, and R⁵ are each independently selected from hydrogen and lower alkyl;
R² is independently selected from Ar or is hydrogen, hydroxy, alkoxy, —NMe₂, —CONR⁹R¹⁰ wherein R⁹ and R¹⁰ are each independently selected from hydrogen, straight or branched alkyl of up to 6 carbon atoms, or R⁹ and R¹⁰ together with the nitrogen atom to which they are linked can form a 5- to 7-membered aliphatic ring which may contain 1 or 2 oxygen or nitrogen atoms, or R² is 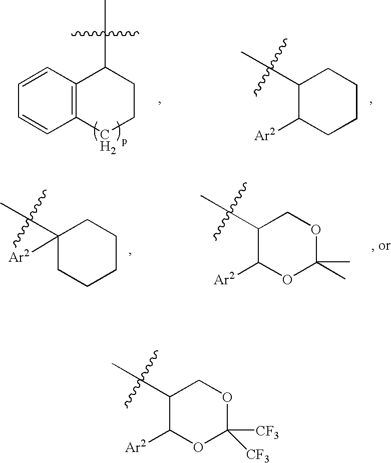
wherein p is 0, 1 or 2 and Ar² is phenyl or pyridyl;
X is a divalent radical derived from any of the following: 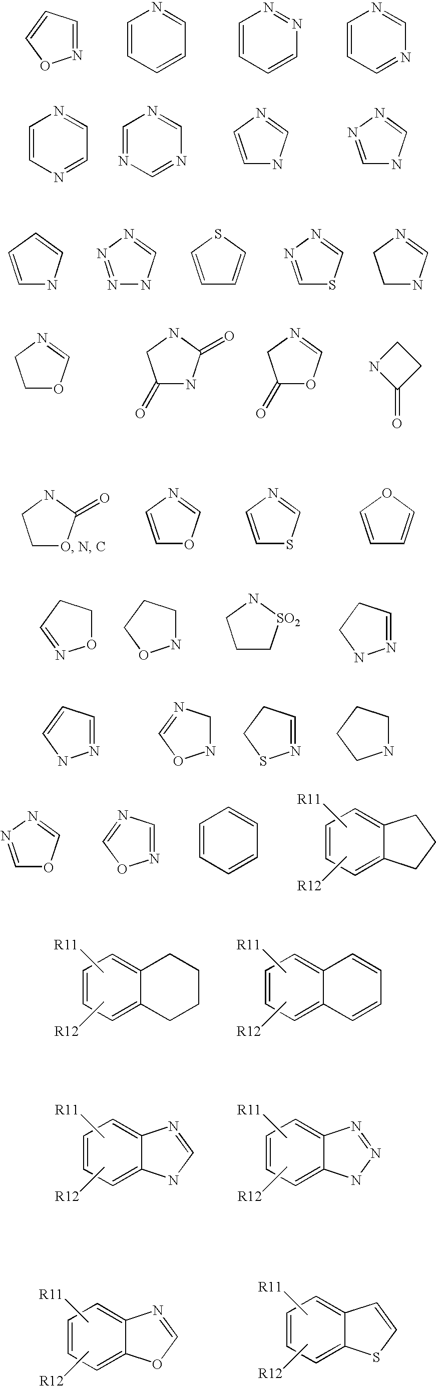 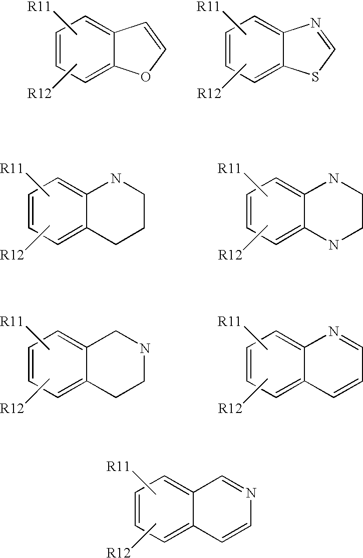
where the ring nitrogen atoms may have lower alkyl groups attached thereto, R¹¹, R¹² are independently selected from H, halogen, hydroxy, alkoxy, acetyl, nitro, cyano, amino, CF₃ and (CH₂)ₜNR¹³R¹⁴ wherein t can be 0 or 1, R¹³ and R¹⁴ are each independently selected from hydrogen, straight or branched alkyl of up to 6 carbon atoms or cycloalkyl of 5 to 7 carbon atoms, containing up to 2 oxygen or nitrogen atoms;
provided that, when Ar¹ is indolyl, then
(i) r is 1 or q is 1, or
(ii) R⁶ forms with R¹ an aliphatic ring of from 3 to 7 atoms which can contain an oxygen or nitrogen atom, or R⁶ together with R¹ is a carbonyl group.

2. A compound of the formula (II), or a pharmaceutically acceptable salt thereof: 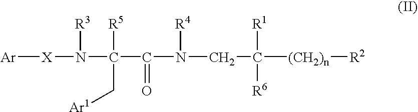
wherein:
n is 0 or 1;
Ar is phenyl or pyridyl which may be unsubstituted or substituted with from 1 to 3 substituents selected from halogen, alkoxy, nitro and cyano;
Ar¹ is independently selected from Ar or is pyridyl-N-oxide or indolyl;
R⁶ forms with R¹ an aliphatic ring of from 3 to 7 atoms which can contain an oxygen or nitrogen atom, or together with R¹ is a carbonyl group;
R² is independently selected from Ar or is hydrogen, hydroxy, alkoxy, dimethylamino, tetrazolyl or —CONR⁹R¹⁰ wherein R⁹ and R¹⁰ are each independently selected from hydrogen or methyl, or R² is any of 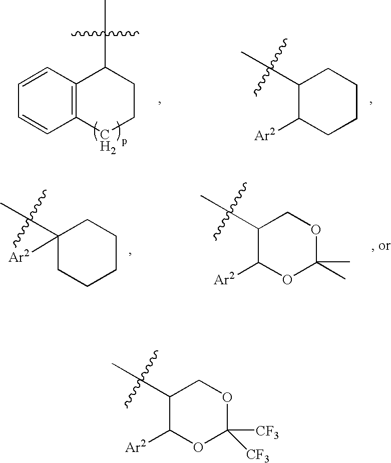
wherein p is 0, 1 or 2, and Ar² is phenyl or pyridyl;
R³, R⁴ and R⁵ are each independently selected from hydrogen and methyl; and
X is selected from: 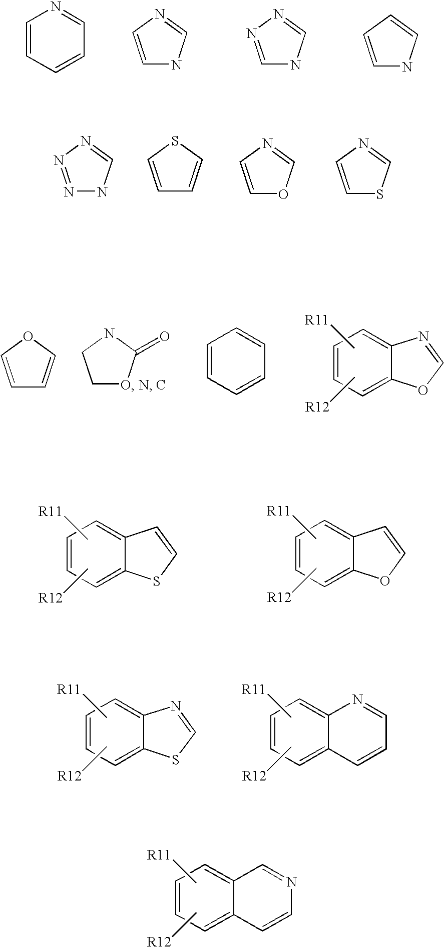
R¹¹ and R¹² being independently selected from H, halogen, hydroxy, alkoxy, acetyl, nitro, cyano, amino, CF₃ and (CH₂)ₜNR¹³R¹⁴ wherein t is 0 or 1 and R¹³ and R¹⁴ are independently selected from hydrogen and methyl.

3. A compound of the formula (IIa) or (IIb): 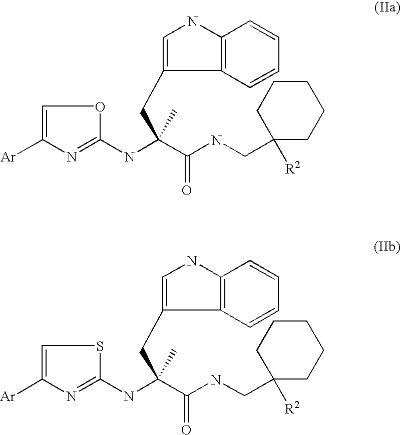
wherein Ar and R² independently represent phenyl or pyridyl which may be unsubstituted or substituted with from 1 to 3 substituents selected from halogen, alkoxy, nitro and cyano, and pharmaceutically acceptable salts thereof.

4. (S)-3-(1H-Indol-3-yl)-N-[1-(5-methoxy-pyridin-2-yl)-cyclohexylmethyl]-2-methyl-2-[4-(4-nitro-phenyl)-oxazol-2-ylamino]-propionamide and its pharmaceutically acceptable salts.

5. Any of the following compounds or a pharmaceutically acceptable salt thereof:
(S)-3-(1H-indol-3-yl)-N-(1-methoxymethyl-cyclohexylmethyl)-2-methyl-2-[4-(4-nitro-phenyl)-oxazol-2-ylamino]-propionamide;
(S)-3-(1H-indol-3-yl)-2-methyl-2-[4-(4-nitro-phenyl)-oxazol-2-ylamino]-N-(2-oxo-2-phenyl-ethyl)-propionamide;
(S)-N-[1-(5-methoxy-pyridin-2-yl)-cyclohexylmethyl]-2-methyl-2-[4-(4-nitro-phenyl)-oxazol-2-ylamino]-3-phenyl-propionamide;
(S)-2-[4-(4-cyano-phenyl)-oxazol-2-ylamino]-3-(1H-indol-3-yl)-N-[1-(5-methoxy-pyridin-2-yl)-cyclohexylmethyl]-2-methyl-propionamide;
(S)-3-(1H-indol-3-yl)-N-[1-(5-methoxy-pyridin-2-yl)-cyclohexylmethyl]-2-methyl-2-(4-phenyl-oxazol-2-ylamino)-propionamide;
(S)-2-(4-ethyl-oxazol-2-ylamino)-3-(1H-indol-3-yl)-N-[1-(5-methoxy-pyridin-2-yl)-cyclohexylmethyl]-2-methyl-propionamide;
(S)-3-(1H-indol-3-yl)-N-[1-(5-methoxy-pyridin-2-yl)-cyclohexylmethyl]-2-methyl-2-[4-(4-nitro-phenyl)-thiazol-2-ylamino]-propionamide;
(S)-2-(benzooxazol-2-ylamino)-3-(1H-indol-3-yl)-2-methyl-N-(1-pyridin-2-yl-cyclohexylmethyl)-propionamide;
(S)-3-(1H-indol-3-yl)-2-methyl-2-(pyridin-4-ylamino)-N-(1-pyridin-2-yl-cyclohexylmethyl)-propionamide;
(S)-3-(1H-indol-3-yl)-2-(isoquinol-4-ylamino)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(pyrimidin-5-ylamino)-propionamide;
(S)-2-(biphenyl-2-ylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-m-tolylamino-propionamide;
(S)-3-(1H-indol-3-yl)-2-methyl-2-(6-phenyl-pyridin-2-ylamino)-N-(1-pyridin-2-yl-cyclohexylmethyl)-propionamide;
(R)-3-phenyl-2-phenylamino-N-(1-pyridin-2-yl-cyclohexylmethyl)-propionamide;
(S)-3-(1H-indol-3-yl)-2-methyl-2-phenylethylamino-N-(1-pyridin-2-yl-cyclohexylmethyl)-propionamide;
(S)-2-[(benzofuran-2-ylmethyl)-amino]-3-(1H-indol-3-yl)-2-methyl-N-(1-pyridin-2-yl-cyclohexylmethyl)-propionamide, and
(S)-3-(1H-indol-3-yl)-2-methyl-2-(4-nitro-benzylamino)-N-(1-pyridin-2-yl-cyclohexylmethyl)-propionamide.

6. A salt of a compound according to any preceding claim which is a hydrochloride, mesylate or sulfate.

7. A method for making a compound of the formula (I) defined in claim 1 in which r is 1, j is 0, q is 1, k is 0 and X is -oxazol-2-yl-, which comprises:
(a) converting a methyl ester of the formula (III) 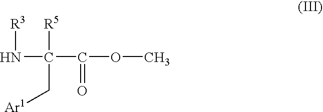
where R³, R⁵ and Ar¹ have the meanings given in claim 1 via a p-nitrophenylcarbamate to a urea of the formula (IV): 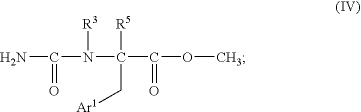
(b) cyclising the urea by reaction with a compound of the formula ArCOCH₂Hal wherein Ar has the meaning given in claim 1 and Hal represents a halogen to give a compound of the formula (V) 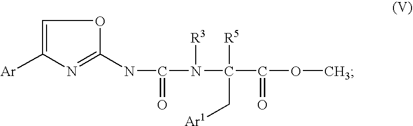
(c) forming an amide bond between the carboxyl group of the compound of formula (V) and an amine of the formula (VI) by removing the methoxy group from the compound of formula (V) and reacting the resulting acid in the presence of O-benzotriazol-1-yl-N,N,N′N′-tetramethyluronium hexafluorophosphate with an amine of the formula (VI) 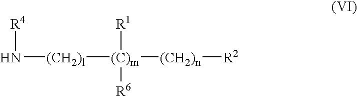
to give the compound of formula (I) and
(d) optionally converting said compound to a pharmaceutically acceptable salt.

8. A method for making a compound of formula (I) as defined in claim 1 in which k is 0, which comprises:
(a) substituting the halogen of a compound of the formula (Ar)ᵣ—(CH₂)ⱼ—(X)_{q}-Hal in which r, j, q, k, Ar and X are as defined in claim 1 and Hal represents a halogen atom by an amino group of a compound of the formula (VII) by reaction in the presence of a base with a copper salt as catalyst 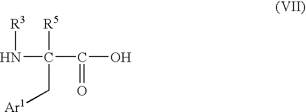
the groups R³, R⁵ and Ar1 being as defined in claim 1;
(b) forming an amide linkage by reacting the resulting acid in the presence of O-benzotriazol-1-yl-N,N,N′N′-tetramethyluronium hexafluorophosphate with an amine of the formula (VI) as defined in claim 7 to give the compound of formula (I); and
(c) optionally converting said compound to an acid addition salt.

9. A method for making a compound of the formula (I) defined in claim 1 in which k is 1, which comprises:
(a) protecting with a protective group the amine group of a compound of formula (VII) as defined in claim 8;
(b) forming an amide linkage by reacting the protected acid in the presence of O-benzotriazol-1-yl-N,N,N′N′-tetramethyluronium hexafluorophosphate with an amine of the formula (VI) as defined in claim 7;
(c) deprotecting the amino group of the resulting amide;
(d) substituting the halogen of a compound of the formula (Ar)ᵣ—(CH₂)ⱼ—(X)_{q}—(CH₂)ₖ-Hal in which r, j, q, Ar and X are as defined in claim 1, k is 1 and Hal represents a halogen atom by an amino group of the deprotected amide by reaction in the presence of a base with a copper salt as catalyst to give the compound of formula (I); and
(e) optionally converting said compound to an acid addition salt.

10. A pharmaceutical composition comprising a therapeutically effective amount of a compound according to any of claims 1-6 in combination with a pharmaceutically acceptable carrier.

11. A method of antagonizing the effects of neuromedin B and/or gastrin-releasing peptide at bombesin receptors which comprises administering a compound according to any of claims 1-6 to a patient.

12. A method of treating sexual dysfunction in a male patient in need of said treatment comprising administering a therapeutically effective amount of a compound according to any of claims 1-6.

13. A method of treating sexual dysfunction characterized by generalized unresponsiveness or ageing-related decline in sexual arousability in a male patient in need of said treatment, comprising administering a therapeutically effective amount of a compound according to any of claims 1-6.

14. Use of a compound of any of claims 1-6 in the manufacture of a medicament for preventing or treating sexual dysfunction in a male patient.

15. Use of a compound of any of claims 1-6 in the manufacture of a medicament for preventing or treating sexual dysfunction characterized by generalized unresponsiveness or ageing-related decline in sexual arousability in a male patient.

16. A method of treating sexual dysfunction in a female patient in need of said treatment comprising administering a therapeutically effective amount of a compound according to any of claims 1-6.

17. A method of treating sexual dysfunction characterized by generalized unresponsiveness or ageing-related decline in sexual arousability in a female patient in need of said treatment, comprising administering a therapeutically effective amount of a compound according to any of claims 1-6.

18. A method of treating sexual dysfunction in a female patient, characterized by hypoactive sexual desire disorders, sexual arousal disorders, orgasmic disorders or anorgasmy, or sexual pain disorders, in need of said treatment comprising administering a therapeutically effective amount of a compound according to any of claims 1-6.

19. Use of a compound of any of claims 1-6 in the manufacture of a medicament for preventing or treating sexual dysfunction in a female patient.

20. Use of a compound of any of claims 1-6 in the manufacture of a medicament for preventing or treating sexual dysfunction characterized by generalized unresponsiveness or ageing-related decline in sexual arousability in a female patient.

21. Use of a compound of any of claims 1-6 in the manufacture of a medicament for preventing or treating sexual dysfunction in female patients characterized by hypoactive sexual desire disorders, sexual arousal disorders, orgasmic disorders or anorgasmy, or sexual pain disorders.

22. A method of treating anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders and pruritus in a patient in need of said treatment comprising administering a therapeutically effective amount of a compound according to any of claims 1-6

23. Use of a compound of any of claims 1-6 in the manufacture of a medicament for preventing or treating anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders and pruritus.

24. Use as claimed in any of claims 14, 15, 19, 20, 21 and 23 wherein the medicament is adapted for oral administration.
